SEQUENCE LISTING (322 sequences)

SEQ ID NO: 1 - DNA, 1510 bp, Artificial sequence
  CD37 specific binding protein
aagcttgccgccatggattttcaagtgcagattttcagcttcctgctaatcagtgcttcagtcataattgccagaggagtcgacatccagatgactcagtctccagcctccctatctgcatctgtgggagagactgtcaccatcacatgtcgaacaagtgaaaatgtttacagttatttggcttggtatcagcagaaacagggaaaatctcctcagctcctggtctcttttgcaaaaaccttagcagaaggtgtgccatcaaggttcagtggcagtggatcaggcacacagttttctctgaagatcagcagcctgcagcctgaagattctggaagttatttctgtcaacatcattccgataatccgtggacgttcggtggaggcaccgaactggagatcaaaggtggcggtggctcgggcggtggtgggtcgggtggcggcggatcgtcagcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttcagatctggagcccaaatcttctgacaaaactcacacatctccaccgtgcccagcacctgaactcttgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtctaga

SEQ ID NO: 2 - protein, 496 aa, Artificial sequence
  CD37 specific binding protein
MDFQVQIFSFLLISASVIIARGVDIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKGGGGSGGGGSGGGGSSAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSDLEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3-4 - skipped (2 entries)

SEQ ID NO: 5 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 493 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggagctctgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 493 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGSSEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagagtcgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 493 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAESKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgaacaagtcaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 493 aa, Artificial sequence
  CD37 specific binding protein
MEAPAQLLFLLLLWLPDTTGEIVLTQSPATLSLSPGERATLSCRTSQNVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgaacaagtgaaagtgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 493 aa, Artificial sequence
  CD37 specific binding protein
MEAPAQLLFLLLLWLPDTTGEIVLTQSPATLSLSPGERATLSCRTSESVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 493 aa, Artificial sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1479 bp, Artificial sequence
  CD37 specific binding protein
atggaagcaccagcgcagcttctcttcctcctgctactctggctcccagataccaccggtgacatccagatgactcagtctccagcctccctatctgcatctgtgggagagactgtcaccatcacatgtcgaacaagtgaaaatgtttacagttatttggcttggtatcagcagaaacagggaaaatctcctcagctcctggtctcttttgcaaaaaccttagcagaaggtgtgccatcaaggttcagtggcagtggatcaggcacacagttttctctgaagatcagcagcctgcagcctgaagattctggaagttatttctgtcaacatcattccgataatccgtggacgttcggtggaggcaccgaactggagatcaaaggtggcggtggctcgggcggtggtgggtcgggtggcggcggagctagcgaggtgcagctggtgcagtctggagcagaggtgaaaaagcccggagagtctctgaggatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 492 aa, Artificial sequence
  CD37 specific binding protein
MEAPAQLLFLLLLWLPDTTGDIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKGGGGSGGGGSGGGGASEVQLVQSGAEVKKPGESLRISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1479 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtagtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGSGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1479 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagccaggtgcagctggtggagtctggtggaggcgtggtccagcctgggaggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASQVQLVESGGGVVQPGRSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1503 bp, Artificial sequence
  CD37 specific binding protein
atggaagcaccagcgcagcttctcttcctcctgctactctggctcccagataccaccggtgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttctggtggcggtggctcgggcggtggtgggtcgggtggcggcggatcaggaggaggcgggagtgctagcgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 500 aa, Artificial sequence
  CD37 specific binding protein
MEAPAQLLFLLLLWLPDTTGAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSGGGGSGGGGSGGGGSGGGGSASEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1488 bp, Artificial sequence
  CD37 specific binding protein
atggattttcaagtgcagattttcagcttcctgctaatcagtgcttcagtcataattgccagaggagtcgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 495 aa, Artificial sequence
  CD37 specific binding protein
MDFQVQIFSFLLISASVIIARGVEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1503 bp, Artificial sequence
  CD37 specific binding protein
atggaagcaccagcgcagcttctcttcctcctgctactctggctcccagataccaccggtgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttctggtggcggtggctcgggcggtggtgggtcgggtggcggcggatcaggaggaggcgggagtgctagcgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - protein, 500 aa, Artificial sequence
  CD37 specific binding protein
MEAPAQLLFLLLLWLPDTTGAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSGGGGSGGGGSGGGGSGGGGSASEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1479 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1479 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagcgcggtccagctgcagcagtctggacctgagtcggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1479 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggagctagccaggtgcagctggtggagtctggtggaggcgtggtccagcctgggaggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASQVQLVESGGGVVQPGRSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1479 bp, Artificial sequence; identical to SEQ ID NO: 33
  CD37 specific binding protein

SEQ ID NO: 36 - protein, 492 aa, Artificial sequence; identical to SEQ ID NO: 22
  CD37 specific binding protein

SEQ ID NO: 37 - DNA, 1476 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggcttggtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 38 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGLVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1476 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggcttggtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGLVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1476 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggctctgtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 42 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGSVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1476 bp, Artificial sequence
  CD37 specific binding protein
atggaagcaccagcgcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtggagtctggtggaggctctgtccagcctggagggtccctgagactctcctgtgcagcctctggattcaccttcagtggctacaatatgaactgggtccgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 492 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVESGGGSVQPGGSLRLSCAASGFTFSGYNMNWVRQMPGKGLEWMGNIDPYYGGTTYNRKFKGQVTISADKSISTAYLQWSSLKASDTAMYYCARSVGPMDYWGRGTLVTVSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1482 bp, Artificial sequence
  CD37 specific binding protein
atggaagccccagctcagcttctcttcctcctgctactctggctcccagataccaccggagaaattgtgttgacacagtctccagccaccctgtctttgtctccaggcgaaagagccaccctctcctgccgagcaagtcaaagtgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 46 - protein, 493 aa, Artificial sequence
  CD37 specific binding protein
MEAPAQLLFLLLLWLPDTTGEIVLTQSPATLSLSPGERATLSCRASQSVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 1500 bp, Artificial sequence
  CD37 specific binding protein
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggcggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactactggggccagggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 48 - protein, 493 aa, Artificial sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDYWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49-50 - skipped (2 entries)

SEQ ID NO: 51 - DNA, 1381 bp, Artificial sequence
  CD37 specific binding protein
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctca

SEQ ID NO: 52 - protein, 493 aa, Artificial sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53-60 - skipped (8 entries)

SEQ ID NO: 61 - protein, 11 aa, Artificial sequence
  CDR
RASENVYSYLA

SEQ ID NO: 62 - protein, 11 aa, Artificial sequence
  CDR
RTSENVYSYLA

SEQ ID NO: 63 - protein, 5 aa, Artificial sequence
  CDR
GYMNM

SEQ ID NO: 64 - protein, 7 aa, Artificial sequence
  CDR
FAKTLAE

SEQ ID NO: 65 - protein, 17 aa, Artificial sequence
  CDR
NIDPYYGGTTYNRKFKG

SEQ ID NO: 66 - protein, 9 aa, Artificial sequence
  CDR
QHHSDNPWT

SEQ ID NO: 67 - protein, 7 aa, Artificial sequence
  CDR
SVGPFDY

SEQ ID NO: 68 - protein, 7 aa, Artificial sequence
  CDR
SVGPFDS

SEQ ID NO: 69 - protein, 7 aa, Artificial sequence
  CDR
SVGPMDY

SEQ ID NO: 70-78 - skipped (9 entries)

SEQ ID NO: 79 - DNA, 1500 bp, Artificial sequence
  CD37 specific binding protein
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgacctctggggcagaggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 80 - protein, 493 aa, Artificial sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDLWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1494 bp, Artificial sequence
  CD37 specific binding protein
aagcttgccgccatggaagccccagctcagcttctcttcctcctgctactctggctcccagataccaccggagaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtgcagtctggagcagaggtgaaaaagcccggagagtctctgaagatttcctgtaagggatccggttactcattcactagctacaatatgaactgggtgcgccagatgcccgggaaaggcctggagtggatgggcaatattgatccttattatggtggtactaactacgcccagaagttccagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 82 - protein, 493 aa, Artificial sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLKISCKGSGYSFTSYNMNWVRQMPGKGLEWMGNIDPYYGGTNYAQKFQGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 1476 bp, Artificial sequence
  CD37 specific binding protein
aagcttgccgccatggaagccccagcgcagcttctcttcctcctgctactctggctcccagataccaccggagaaattgtgttgacacagtctccagccaccctgtctttgtctccaggcgaaagagccaccctctcctgccgagcaagtgagaatgtttacagctacttagcctggtaccaacagaaacctggccaggctcctaggctcctcatctattttgcaaaaaccttagcagaagggattccagccagattcagtggcagtggttccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggagcggaggaggagctagcgaggtgcagctggtgcagtctggagcagaggtgaaaaagcccggagagtctctgaagatttcctgtaagggatccggttactcattcactggctacaatatgaactgggtgcgccagatgcccgggaaaggcctcgaatggatgggcaatattgatccttattatggtggtactacctacaaccggaagttcaagggccaggtcactatctccgccgacaagtccatcagcaccgcctacctgcaaggagcagcctgaaggcctcggacaccgccatgtattactgtgcacgctcagtcggccctttcgactcctggggccagggcaccctggtcactgtctcgagttgtccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactctaga

SEQ ID NO: 84 - protein, 485 aa, Artificial sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGSGGGASEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1494 bp, Artificial sequence
  CD37 specific binding protein
aagcttgccgccatggaagccccagctcagcttctcttcctcctgctactctggctcccagataccaccggagaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtgggaccggtgaggtgcagctggtgcagtctggagcagaggtgaaaaagcccggagagtctctgaagatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 86 - protein, 493 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1494 bp, Artificial sequence
  CD37 specific binding protein
aagcttgccgccatggaagccccagctcagcttctcttcctcctgctactctggctcccagataccaccggtgaaattgtgttgacacagtctccagccaccctgtctttgtctccaggcgaaagagccaccctctcctgccgaacaagtgaaaatgtttacagctacttagcctggtaccaacagaaacctggccaggctcctaggctcctcatctattttgcaaaaaccttagcagaaggaattccagccaggttcagtggcagtggatccgggacagacttcactctcaccatcagcagcctagagcctgaagattttgcagtttattactgtcaacatcattccgataatccgtggacattcggccaagggaccaaggtggaaatcaaaggtggcggtggctcgggcggtggtggatctggaggaggtggggctagcgaggtgcagctggtgcagtctggagcagaggtgaaaaagcccggagagtctctgaggatttcctgtaagggatccggttactcattcactggctacaatatgaactgggtgcgccagatgcccgggaaaggcctggagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctatggactactggggccgcggcaccctggtcactgtctcctctgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 88 - protein, 493 aa, Artificial sequence
  CD37 specific binding protein
MEAPAQLLFLLLLWLPDTTGEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLRISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 45 bp, Artificial sequence
  Hinge region
gagcccaaatcttgtgacaaaactcacacatgtccaccgtgccca

SEQ ID NO: 90 - protein, 15 aa, Artificial sequence
  Hinge region
EPKSCDKTHTCPPCP

SEQ ID NO: 91 - DNA, 45 bp, Artificial sequence
  Hinge region
gagcccaaatcttctgacaaaactcacacatgtccaccgtgccca

SEQ ID NO: 92 - protein, 15 aa, Artificial sequence
  Hinge region
EPKSSDKTHTCPPCP

SEQ ID NO: 93 - DNA, 45 bp, Artificial sequence
  Hinge region
gagcccaaatcttctgacaaaactcacacatgtccaccgtgctca

SEQ ID NO: 94 - protein, 15 aa, Artificial sequence
  Hinge region
EPKSSDKTHTCPPCS

SEQ ID NO: 95 - DNA, 45 bp, Artificial sequence
  Hinge region
gagcccaaatcttgtgacaaaactcacacatgtccaccgagctca

SEQ ID NO: 96 - protein, 15 aa, Artificial sequence
  Hinge region
EPKSSDKTHTCPPSS

SEQ ID NO: 97 - DNA, 45 bp, Artificial sequence
  Hinge region
gagcccaaatcttctgacaaaactcacacatctccaccgagccca

SEQ ID NO: 98 - protein, 15 aa, Artificial sequence
  Hinge region
EPKSSDKTHTSPPSP

SEQ ID NO: 99 - DNA, 45 bp, Artificial sequence
  Hinge region
gagcccaaatcttctgacaaaactcacacatctccaccgagctca

SEQ ID NO: 100 - protein, 15 aa, Artificial sequence
  Hinge region
EPKSSDKTHTSPPSS

SEQ ID NO: 101 - DNA, 45 bp, Artificial sequence
  Hinge region
gagcccaaatcttgtgacaaaactcacacatctccaccgtgccca

SEQ ID NO: 102 - protein, 15 aa, Artificial sequence
  Hinge region
EPKSCDKTHTSPPCP

SEQ ID NO: 103 - DNA, 45 bp, Artificial sequence
  Hinge region
gagcccaaatcttgtgacaaaactcacacatctccaccgtgctca

SEQ ID NO: 104 - protein, 15 aa, Artificial sequence
  Hinge region
EPKSCDKTHTSPPCS

SEQ ID NO: 105 - DNA, 45 bp, Artificial sequence
  Hinge region
gagcccaaatcttctgacaaaactcacacatctccaccgtgccca

SEQ ID NO: 106 - protein, 15 aa, Artificial sequence
  Hinge region
EPKSSDKTHTSPPCP

SEQ ID NO: 107 - DNA, 45 bp, Artificial sequence
  Hinge region
gagcccaaatcttctgacaaaactcacacatctccaccgtgctca

SEQ ID NO: 108 - protein, 15 aa, Artificial sequence
  Hinge region
EPKSSDKTHTSPPCS

SEQ ID NO: 109 - DNA, 45 bp, Artificial sequence
  Hinge region
gagcccaaatcttgtgacaaaactcacacatctccaccgagccca

SEQ ID NO: 110 - protein, 15 aa, Artificial sequence
  Hinge region
EPKSCDKTHTSPPSP

SEQ ID NO: 111 - DNA, 45 bp, Artificial sequence
  Hinge region
gagcccaaatcttgtgacaaaactcacacatctccaccgagctca

SEQ ID NO: 112 - protein, 15 aa, Artificial sequence
  Hinge region
EPKSCDKTHTSPPSS

SEQ ID NO: 113-114 - skipped (2 entries)

SEQ ID NO: 115 - protein, 19 aa, Artificial sequence
  Hinge region
VPSTPPTPSPSTPPTPSPS

SEQ ID NO: 116 - protein, 6 aa, Artificial sequence
  Hinge region
VPPPPP

SEQ ID NO: 117 - DNA, 186 bp, Artificial sequence
  Hinge region
gagctcaaaactcctctcggggatacgacccatacgtgtccccgctgtcctgaaccgaagtcctgcgatacgcctccgccatgtccacggtgcccagagcccaaatcatgcgatacgcccccaccgtgtccccgctgtcctgaaccaaagtcatgcgataccccaccaccatgtccaagatgccca

SEQ ID NO: 118 - protein, 62 aa, Artificial sequence
  Hinge region
ELKTPLGDTTHTCPRCPEPKSCDTPPPCPRCPEPKSCDTPPPCPRCPEPKSCDTPPPCPRCP

SEQ ID NO: 119 - DNA, 45 bp, Artificial sequence
  Hinge region
gagcccaaatcttctgacacacctcccccatgcccacggtgcccc

SEQ ID NO: 120 - protein, 15 aa, Artificial sequence
  Hinge region
EPKSSDTPPPCPRCP

SEQ ID NO: 121 - DNA, 45 bp, Artificial sequence
  Hinge region
gagcccaaatcttgtgacacacctcccccatccccacggtcccca

SEQ ID NO: 122 - protein, 15 aa, Artificial sequence
  Hinge region
EPKSCDTPPPSPRSP

SEQ ID NO: 123 - DNA, 45 bp, Artificial sequence
  Hinge region
gagcccaaatcttctgacacacctcccccatccccacggtcccca

SEQ ID NO: 124 - protein, 15 aa, Artificial sequence
  Hinge region
EPKSSDTPPPSPRSP

SEQ ID NO: 125 - DNA, 45 bp, Artificial sequence
  Hinge region
gagcccaaatcttgtgacacacctcccccatccccacggtgccca

SEQ ID NO: 126 - protein, 15 aa, Artificial sequence
  Hinge region
EPKSCDTPPPSPRCP

SEQ ID NO: 127 - protein, 58 aa, Artificial sequence
  Hinge region
ESPKAQASSVPTAQPQAEGSLAKATTAPATTRNTGRGGEEKKKEKEKEEQEERETKTP

SEQ ID NO: 128 - protein, 11 aa, Artificial sequence
  CDR
RTSQNVYSYLA

SEQ ID NO: 129 - protein, 11 aa, Artificial sequence
  CDR
RTSESVYSYLA

SEQ ID NO: 130 - protein, 11 aa, Artificial sequence
  CDR
RASQSVYSYLA

SEQ ID NO: 131 - protein, 11 aa, Artificial sequence
  CDR
RASQSVSSYLA

SEQ ID NO: 132 - protein, 11 aa, Artificial sequence
  CDR
RASQSVSYYLA

SEQ ID NO: 133 - protein, 5 aa, Artificial sequence
  CDR
SYMNM

SEQ ID NO: 134 - protein, 5 aa, Artificial sequence
  CDR
SYWIG

SEQ ID NO: 135 - protein, 7 aa, Artificial sequence
  CDR
AASSLQS

SEQ ID NO: 136 - protein, 7 aa, Artificial sequence
  CDR
GASTRAT

SEQ ID NO: 137 - protein, 7 aa, Artificial sequence
  CDR
DASNRAT

SEQ ID NO: 138 - protein, 17 aa, Artificial sequence
  CDR
IIYPGDSDTRYSPSFQG

SEQ ID NO: 139 - protein, 17 aa, Artificial sequence
  CDR
RIDPSDSYTNYSPSFQG

SEQ ID NO: 140 - protein, 30 aa, Artificial sequence
  Framework region
QVQLVQSGAEVKKPGASVKVSCKASGYTFT

SEQ ID NO: 141 - protein, 30 aa, Artificial sequence
  Framework region
QVQLVQSGAEVKKPGSSVKVSCKASGGTFS

SEQ ID NO: 142 - skipped

SEQ ID NO: 143 - protein, 30 aa, Artificial sequence
  Framework region
EVQLVQSGAEVKKPGATVKISCKVSGYTFT

SEQ ID NO: 144 - protein, 30 aa, Artificial sequence
  Framework region
EVQLVQSGAEVKKPGESLKISCKGSGYSFT

SEQ ID NO: 145 - protein, 30 aa, Artificial sequence
  Framework region
EVQLVQSGAEVKKPGESLRISCKGSGYSFT

SEQ ID NO: 146 - protein, 30 aa, Artificial sequence
  Framework region
QVQLVQSGSELKKPGASVKVSCKASGYTFT

SEQ ID NO: 147 - protein, 14 aa, Artificial sequence
  Framework region
WVRQAPGQGLEWMG

SEQ ID NO: 148-149 - skipped (2 entries)

SEQ ID NO: 150 - protein, 14 aa, Artificial sequence
  Framework region
WVQQAPGKGLEWMG

SEQ ID NO: 151 - protein, 14 aa, Artificial sequence
  Framework region
WVRQMPGKGLEWMG

SEQ ID NO: 152-153 - skipped (2 entries)

SEQ ID NO: 154 - protein, 32 aa, Artificial sequence
  Framework region
RVTMTTDTSTSTAYMELRSLRSDDTAVYYCAR

SEQ ID NO: 155 - protein, 32 aa, Artificial sequence
  Framework region
RVTITADESTSTAYMELSSLRSEDTAVYYCAR

SEQ ID NO: 156 - protein, 32 aa, Artificial sequence
  Framework region
RVTITADKSTSTAYMELSSLRSEDTAVYYCAR

SEQ ID NO: 157 - protein, 32 aa, Artificial sequence
  Framework region
RVTITADTSTDTAYMELSSLRSEDTAVYYCAT

SEQ ID NO: 158 - protein, 32 aa, Artificial sequence
  Framework region
QVTISADKSISTAYLQWSSLKASDTAMYYCAR

SEQ ID NO: 159 - protein, 32 aa, Artificial sequence
  Framework region
HVTISADKSISTAYLQWSSLKASDTAMYYCAR

SEQ ID NO: 160 - protein, 32 aa, Artificial sequence
  Framework region
RFVFSLDTSVSTAYLQISSLKAEDTAVYYCAR

SEQ ID NO: 161 - protein, 11 aa, Artificial sequence
  Framework region
WGQGTLVTVSS

SEQ ID NO: 162 - protein, 11 aa, Artificial sequence
  Framework region
WGRGTLVTVSS

SEQ ID NO: 163 - protein, 11 aa, Artificial sequence
  Framework region
WGQGTMVTVSS

SEQ ID NO: 164-167 - skipped (4 entries)

SEQ ID NO: 168 - protein, 11 aa, Artificial sequence
  Framework region
WGQGTTVTVSS

SEQ ID NO: 169 - protein, 11 aa, Artificial sequence
  Framework region
WGKGTTVTVSS

SEQ ID NO: 170 - protein, 23 aa, Artificial sequence
  Framework region
EIVMTQSPATLSVSPGERATLSC

SEQ ID NO: 171 - protein, 23 aa, Artificial sequence
  Framework region
EIVLTQSPATLSLSPGERATLSC

SEQ ID NO: 172 - protein, 23 aa, Artificial sequence
  Framework region
DIQMTQSPSSLSASVGDRVTITC

SEQ ID NO: 173-174 - skipped (2 entries)

SEQ ID NO: 175 - protein, 23 aa, Artificial sequence
  Framework region
NIQMTQSPSAMSASVGDRVTITC

SEQ ID NO: 176 - skipped

SEQ ID NO: 177 - protein, 23 aa, Artificial sequence
  Framework region
AIQLTQSPSSLSASVGDRVTITC

SEQ ID NO: 178 - protein, 23 aa, Artificial sequence
  Framework region
DIQLTQSPSFLSASVGDRVTITC

SEQ ID NO: 179 - protein, 23 aa, Artificial sequence
  Framework region
AIRMTQSPFSLSASVGDRVTITC

SEQ ID NO: 180 - protein, 23 aa, Artificial sequence
  Framework region
AIQMTQSPSSLSASVGDRVTITC

SEQ ID NO: 181 - protein, 23 aa, Artificial sequence
  Framework region
DIQMTQSPSTLSASVGDRVTITC

SEQ ID NO: 182 - protein, 15 aa, Artificial sequence
  Framework region
WYQQKPGQAPRLLIY

SEQ ID NO: 183 - skipped

SEQ ID NO: 184 - protein, 15 aa, Artificial sequence
  Framework region
WYQQKPGKAPKLLIY

SEQ ID NO: 185 - protein, 15 aa, Artificial sequence
  Framework region
WYQQKPGKVPKLLIY

SEQ ID NO: 186 - protein, 15 aa, Artificial sequence
  Framework region
WYQQKPGKAPKRLIY

SEQ ID NO: 187 - protein, 15 aa, Artificial sequence
  Framework region
WFQQKPGKVPKHLIY

SEQ ID NO: 188 - protein, 15 aa, Artificial sequence
  Framework region
WFQQKPGKAPKSLIY

SEQ ID NO: 189-190 - skipped (2 entries)

SEQ ID NO: 191 - protein, 15 aa, Artificial sequence
  Framework region
WYQQKPAKAPKLFIY

SEQ ID NO: 192-193 - skipped (2 entries)

SEQ ID NO: 194 - protein, 32 aa, Artificial sequence
  Framework region
GIPARFSGSGSGTEFTLTISSLQSEDFAVYYC

SEQ ID NO: 195 - protein, 32 aa, Artificial sequence
  Framework region
GIPARFSGSGSGTDFTLTISSLEPEDFAVYYC

SEQ ID NO: 196 - protein, 32 aa, Artificial sequence
  Framework region
GVPSRFSGSGSGTDFTLTISSLQPEDFATYYC

SEQ ID NO: 197 - protein, 32 aa, Artificial sequence
  Framework region
GVPSRFSGSGSGTDFTLTISSLQPEDVATYYC

SEQ ID NO: 198 - protein, 32 aa, Artificial sequence
  Framework region
GVPSRFSGSGSGTEFTLTISSLQPEDFATYYC

SEQ ID NO: 199-202 - skipped (4 entries)

SEQ ID NO: 203 - protein, 32 aa, Artificial sequence
  Framework region
GVPSRFSGSGSGTDYTLTISSLQPEDFATYYC

SEQ ID NO: 204 - skipped

SEQ ID NO: 205 - protein, 32 aa, Artificial sequence
  Framework region
GVPSRFSGSGSGTEFTLTISSLQPDDFATYYC

SEQ ID NO: 206 - protein, 10 aa, Artificial sequence
  Framework region
FGQGTKVEIK

SEQ ID NO: 207 - protein, 10 aa, Artificial sequence
  Framework region
FGQGTKLEIK

SEQ ID NO: 208 - protein, 10 aa, Artificial sequence
  Framework region
FGPGTKVDIK

SEQ ID NO: 209 - protein, 10 aa, Artificial sequence
  Framework region
FGGGTKVEIK

SEQ ID NO: 210 - protein, 10 aa, Artificial sequence
  Framework region
FGQGTRLEIK

SEQ ID NO: 211-212 - skipped (2 entries)

SEQ ID NO: 213 - protein, 7 aa, Artificial sequence
  CDR
SVGPMDV

SEQ ID NO: 214 - skipped

SEQ ID NO: 215 - protein, 7 aa, Artificial sequence
  CDR
SVGPFDP

SEQ ID NO: 216 - protein, 7 aa, Artificial sequence
  CDR
SVGPFQH

SEQ ID NO: 217 - protein, 7 aa, Artificial sequence
  CDR
SVGPFDV

SEQ ID NO: 218 - protein, 7 aa, Artificial sequence
  CDR
SVGPFDI

SEQ ID NO: 219 - protein, 7 aa, Artificial sequence
  CDR
SVGPFDL

SEQ ID NO: 220 - skipped

SEQ ID NO: 221 - DNA, 1530 bp, Artificial sequence
  CD37 specific binding protein
aagcttgccgccatggaagccccagctcagcttctcttcctcctgctactctggctcccagataccaccggagaggtgcagctggtgcagtctggagcagaggtgaaaaagcccggagagtctctgaagatttcctgtaagggctccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgggggtggaggctctggtggcggtggctctggcggaggtggatccggtggcggcggatctggcgggggtggctctgaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggctccgggacagacttcactctcaccatcagcagcctagagcctgaagattttgcagtttattactgtcaacatcattccgataatccgtggacattcggccaagggaccaaggtggaaatcaaaggtgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 222 - protein, 503 aa, Artificial sequence
  CD37 specific binding protein
MEAPAQLLFLLLLWLPDTTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 23 aa, Artificial Sequence
  Linker sequence
MDFQVQIFSFLLISASVIIARGV

SEQ ID NO: 224 - protein, 20 aa, Artificial Sequence
  Linker sequence
MEAPAQLLFLLLLWLPDTTG

SEQ ID NO: 225 - protein, 16 aa, Artificial Sequence
  Linker sequence
GGGGSGGGGSGGGGSS

SEQ ID NO: 226 - protein, 16 aa, Artificial Sequence
  Linker sequence
GGGGSGGGGSGGGGAS

SEQ ID NO: 227 - protein, 16 aa, Artificial Sequence
  Linker sequence
GGGGSGGSGSGGGGAS

SEQ ID NO: 228 - protein, 16 aa, Artificial Sequence
  Linker sequence
GGGGSGGGGSGGGGTG

SEQ ID NO: 229 - protein, 25 aa, Artificial Sequence
  Linker sequence
GGGGSGGGGSGGGGSGGGGSGGGGS

SEQ ID NO: 230 - protein, 5 aa, Artificial Sequence
  Hinge sequence
CPPCP

SEQ ID NO: 231 - protein, 16 aa, Artificial Sequence
  Hinge sequence
SEPKSSDKTHTSPPCP

SEQ ID NO: 232 - protein, 17 aa, Artificial Sequence
  Hinge sequence
DLEPKSSDKTHTSPPCP

SEQ ID NO: 233 - protein, 17 aa, Artificial Sequence
  Hinge sequence
DQEPKSSDKTHTSPPCP

SEQ ID NO: 234 - protein, 18 aa, Artificial Sequence
  Hinge sequence
GDQEPKSSDKTHTSPPCP

SEQ ID NO: 235 - protein, 18 aa, Artificial Sequence
  Hinge sequence
GSSEPKSSDKTHTSPPCP

SEQ ID NO: 236 - protein, 107 aa, Artificial Sequence
  variable light chain region
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIK

SEQ ID NO: 237 - protein, 107 aa, Artificial Sequence
  variable light chain region
EIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIK

SEQ ID NO: 238 - protein, 107 aa, Artificial Sequence
  variable light chain region
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIK

SEQ ID NO: 239 - protein, 107 aa, Artificial Sequence
  variable light chain region
EIVLTQSPATLSLSPGERATLSCRTSQNVYSYLAWYQQKPGQAPRLLIYFAKTLAEGIPARFSGSGSGTDFTLTISSLEPEDFAVYYCQHHSDNPWTFGQGTKVEIK

SEQ ID NO: 240 - protein, 107 aa, Artificial Sequence
  variable light chain region
EIVLTQSPATLSLSPGERATLSCRTSESVYSYLAWYQQKPGQAPRLLIYFAKTLAEGIPARFSGSGSGTDFTLTISSLEPEDFAVYYCQHHSDNPWTFGQGTKVEIK

SEQ ID NO: 241 - protein, 116 aa, Artificial Sequence
  variable heavy chain region
AVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSS

SEQ ID NO: 242 - protein, 116 aa, Artificial Sequence
  variable heavy chain region
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGQGTLVTVSS

SEQ ID NO: 243 - protein, 116 aa, Artificial Sequence
  variable heavy chain region
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDVWGQGTLVTVSS

SEQ ID NO: 244 - protein, 116 aa, Artificial Sequence
  variable heavy chain region
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDYWGQGTLVTVSS

SEQ ID NO: 245 - protein, 116 aa, Artificial Sequence
  variable heavy chain region
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSS

SEQ ID NO: 246 - protein, 217 aa, Artificial Sequence
  human IgG1 CH2 and CH3 regions
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 473 aa, Artificial Sequence
  CD37 specific binding protein
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKGGGGSGGGGSGGGGSSAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSDLEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 473 aa, Artificial Sequence
  CD37 specific binding protein
EIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDYWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 480 aa, Artificial Sequence
  CD37 specific binding protein
AVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSGGGGSGGGGSGGGGSGGGGSASEIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 472 aa, Artificial Sequence
  CD37 specific binding protein
EIVLTQSPATLSLSPGERATLSCRTSENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASAVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 483 aa, Artificial Sequence
  CD37 specific binding protein
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMDVWGQGTTVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 15 aa, Artificial Sequence
  Hinge region
EPKSCDKTHTCPPSP

SEQ ID NO: 256 - protein, 15 aa, Artificial Sequence
  Hinge region
EPKSCDKTHTCPPSS

SEQ ID NO: 257 - protein, 15 aa, Artificial Sequence
  Hinge region
EPKSSDKTHTCPPSP

SEQ ID NO: 258 - protein, 15 aa, Artificial Sequence
  Hinge region
EPKSCDTPPPCPRCP

SEQ ID NO: 259 - protein, 15 aa, Artificial Sequence
  Hinge region
EPKSCDTPPPCPRSP

SEQ ID NO: 260 - protein, 15 aa, Artificial Sequence
  Hinge region
EPKSCDTPPPSPRCP

SEQ ID NO: 261 - protein, 15 aa, Artificial Sequence
  Hinge region
EPKSCDTPPPCPRSP

SEQ ID NO: 262 - protein, 493 aa, Artificial Sequence
  CD37 specific binding protein
MEAPAQLLFLLLLWLPDTTGEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGASEVQLVQSGAEVKKPGESLKISCKGSGYSFTSYNMNWVRQMPGKGLEWMGNIDPYYGGTGYAQKFQGQVTISADKSISTAYLQWSSLKASDTAMYYCARSVGPMDYWGRGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 10 aa, Homo sapiens
EPKSCDKTHT

SEQ ID NO: 264 - protein, 4 aa, Homo sapiens
CPPC

SEQ ID NO: 265 - protein, 4 aa, Homo sapiens
GTCY

SEQ ID NO: 266 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDPWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPMEHWGQGTLVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDVWGQGTMVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 269 - protein, 473 aa, Artificial Sequence
  CD37 specific binding protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGGGGSGGGGSGGGGTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDIWGQGTMVTVSSDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 7 aa, Homo sapiens
ERKCCVE

SEQ ID NO: 271 - protein, 12 aa, Homo sapiens
ELKTPLGDTTHT

SEQ ID NO: 272 - protein, 10 aa, Homo sapiens
EPKSCDTPPP

SEQ ID NO: 273 - protein, 7 aa, Homo sapiens
ESKYGPP

SEQ ID NO: 274 - protein, 5 aa, Homo sapiens
CPPCP

SEQ ID NO: 275 - protein, 5 aa, Homo sapiens
CPRCP

SEQ ID NO: 276 - protein, 5 aa, Homo sapiens
CPSCP

SEQ ID NO: 277 - protein, 17 aa, Homo sapiens
ELKTPLGDTTHTCPRCP

SEQ ID NO: 278 - protein, 15 aa, Homo sapiens
EPKSCDTPPPCPRCP

SEQ ID NO: 279 - protein, 34 aa, Homo sapiens
ESPKAQASSVPTAQPQAEGSLAKATTAPATTRNT

SEQ ID NO: 280 - protein, 24 aa, Homo sapiens
GRGGEEKKKEKEKEEQEERETKTP

SEQ ID NO: 281 - protein, 19 aa, Homo sapiens
VPSTPPTPSPSTPPTPSPS

SEQ ID NO: 282 - protein, 6 aa, Homo sapiens
VPPPPP

SEQ ID NO: 283 - protein, 107 aa, Homo sapiens
VCSRDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 284 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 285 - protein, 110 aa, Homo sapiens
  DOMAIN (1)...(110): Wild type human CH2 domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 286 - protein, 101 aa, Homo sapiens
  DOMAIN (1)...(101): Wild type human CH2 domain
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 287 - protein, 101 aa, Homo sapiens
  DOMAIN (1)...(101): Wild type human CH2 domain
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 288 - protein, 108 aa, Homo sapiens
  DOMAIN (1)...(108): Wild type human CH2 domain
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 289 - protein, 107 aa, Homo sapiens
  DOMAIN (1)...(107): Wild type human CH2 domain
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 290 - protein, 109 aa, Homo sapiens
  DOMAIN (1)...(109): Wild type human CH2 domain
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 291 - protein, 110 aa, Homo sapiens
  DOMAIN (1)...(110): Wild type human CH2 domain
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 292 - protein, 110 aa, Homo sapiens
  DOMAIN (1)...(110): Wild type human CH2 domain
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 293 - protein, 112 aa, Homo sapiens; identical to SEQ ID NO: 284
  DOMAIN (1)...(112): Wild type human CH2 domain

SEQ ID NO: 294 - protein, 107 aa, Homo sapiens
  DOMAIN (1)...(107): Wild type human CH3 domain
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - protein, 131 aa, Homo sapiens
  DOMAIN (1)...(131): Wild type human CH3 domain
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 296 - protein, 131 aa, Homo sapiens; identical to SEQ ID NO: 295
  DOMAIN (1)...(131): Wild type human CH3 domain

SEQ ID NO: 297 - protein, 117 aa, Homo sapiens
  DOMAIN (1)...(117): Wild type human CH3 domain
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 298 - protein, 108 aa, Homo sapiens
  DOMAIN (1)...(108): Wild type human CH3 domain
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 299 - protein, 107 aa, Homo sapiens
  DOMAIN (1)...(107): Wild type human CH3 domain
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 300 - protein, 107 aa, Homo sapiens
  DOMAIN (1)...(107): Wild type human CH3 domain
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 301 - protein, 107 aa, Homo sapiens
  DOMAIN (1)...(107): Wild type human CH3 domain
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 302 - protein, 106 aa, Homo sapiens
  DOMAIN (1)...(106): Wild type human CH3 domain
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 303 - protein, 110 aa, Homo sapiens
  DOMAIN (1)...(110): Wild type human CH4 domain
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 304 - protein, 131 aa, Homo sapiens
  DOMAIN (1)...(131): Wild type human CH4 domain
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 305 - protein, 110 aa, Artificial Sequence
  Human IgG1 CH2 with L235A, E318A, K320A and K322A substitutions
APELAGGPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAK

SEQ ID NO: 306 - protein, 110 aa, Artificial Sequence
  Human IgG1 CH2 with L234A, L235A, G237A, E318A, K320A and K322A
APEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAK

SEQ ID NO: 307 - protein, 214 aa, Artificial Sequence
  G28-1 Kappa mAB (CAS068) protein
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 449 aa, Artificial Sequence
  G28-1 Heavy mAB (CAS069) protein
AVQLQQSGPESEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPSGAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 214 aa, Artificial Sequence
  CAS024 Kappa mAB (CAS066) protein
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 449 aa, Artificial Sequence
  CAS024 Heavy mAB (CAS067) protein
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPSGAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 11 aa, Artificial Sequence
  CDR
KASQDVSTAVA

SEQ ID NO: 312 - protein, 10 aa, Artificial Sequence
  CDR
RASSSIVYMH

SEQ ID NO: 313 - protein, 10 aa, Artificial Sequence
  CDR
GYSFTDFNMY

SEQ ID NO: 314 - protein, 10 aa, Artificial Sequence
  CDR
GFTFRSYGMS

SEQ ID NO: 315 - protein, 7 aa, Artificial Sequence
  CDR
WASTRHT

SEQ ID NO: 316 - protein, 7 aa, Artificial Sequence
  CDR
DTSKLAS

SEQ ID NO: 317 - protein, 17 aa, Artificial Sequence
  CDR
YIDPYNGDTTYNQKFKG

SEQ ID NO: 318 - protein, 16 aa, Artificial Sequence
  CDR
SINSDGGSTYYPDVKG

SEQ ID NO: 319 - protein, 9 aa, Artificial Sequence
  CDR
QQHYSTPLT

SEQ ID NO: 320 - protein, 9 aa, Artificial Sequence
  CDR
HQRSSYPTT

SEQ ID NO: 321 - protein, 9 aa, Artificial Sequence
  CDR
GPNWVAMDY

SEQ ID NO: 322 - protein, 13 aa, Artificial Sequence
  CDR
GGALIVTSDAMDY

(19) United States
(12) Patent Application Publication
(10) Pub. No.: US 2010/0135900 A1
(43) Pub. Date: Jun. 3, 2010

(54) CD37 IMMUNOTHERAPEUTIC COMBINATION THERAPIES AND USES THEREOF

(75) Inventors: CHARLES G. CERVENY, SEATTLE, WA (US); PETER A. THOMPSON, BELLEVUE, WA (US)

Correspondence Address: Seed Intellectual Property Law Group PLLC, 701 Fifth Avenue, Suite 5400, Seatle, WA, 98104, US

(73) Assignee: TRUBION PHARMACEUTICALS, INC., SEATTLE, WA (US)

(21) Appl. No.: 12/618,509

(22) Filed: Nov. 13, 2009

Related U.S. Application Data
(60) Provisional application No. 61/114,385, filed on Nov. 13, 2008.

(51) Int. Cl. A61K 51/00; A61K 31/436; A61K 31/12; A61K 31/60; A61K 39/395; A61K 38/00; A61K 31/415

(52) U.S. Cl. 424/1.11; 514/291; 514/685; 514/159; 424/130.1; 424/133.1; 514/12; 514/394

(57) ABSTRACT

The present disclosure provides methods for using CD37-specific binding molecules (such as a CD37-specific SMIP or antibody) in combination with mTOR inhibitors (such as rapamycin and derivatives or analogues thereof) or phosphatidylinositol 3-kinase (PI3K) inhibitors (such as p110δ-specific inhibitors or the like), which can be done concurrently or sequentially, to treat or prevent a B-cell related hyperproliferative disease, such as a lymphoma, carcinoma, myeloma, or the like.

### CROSS-REFERENCE TO RELATED APPLICATION

This application claims the benefit under 35 U.S.C. §119(e) of U.S. Provisional Patent Application No. 61/114,385 filed Nov. 13, 2008, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910180_{—}418_SEQUENCE_LISTING.txt. The text file is 325 KB, was created on Nov. 13, 2009, and is being submitted electronically via EFS-Web, concurrent with the filing of the specification.

### BACKGROUND

1. Technical Field

The present disclosure generally provides compositions and methods for treating B-cell disorders and, more specifically, to the use of CD37-specific binding molecules in combination with mTOR or phosphatidylinositol 3-kinase (PI3K) inhibitors, including compositions thereof, that act synergistically in treating or preventing B-cell related hyperproliferative diseases, such as lymphoma, carcinoma, myeloma, or the like.

2. Description of the Related Art

The human immune system generally protects the body from invading foreign substances and pathogens. One component of the immune system is B lymphocytes, also referred to as B-cells, which produce antibodies that protect the body by binding to, and in some cases mediating destruction of, a foreign substance or pathogen. In some instances, however, the immune system functions can go awry and disease results. For example, there are numerous cancers, autoimmune diseases, and inflammatory diseases that involve uncontrolled proliferation of B-cells.

B-cells can be identified by molecules on their cell surface, such as CD37. CD37 is a heavily glycosylated 40-52 kDa protein that belongs to the tetraspanin transmembrane family of cell surface antigens, which is highly expressed on normal antibody-producing B-cells but not on pre-B-cells or plasma cells. In addition to normal B-cells, almost all malignancies of B-cell origin are positive for CD37 expression, including chronic lymphocytic leukemia (CLL), non-Hodgkins lymphoma (NHL), and hairy cell leukemia (Moore et al., J. Pathol. 152:13 (1987); Merson and Brochier, Immunol. Lett. 19:269 (1988); and Faure et al., Am. J. Dermatopathol. 12:122 (1990)).

A few CD37 specific immunotherapies have been developed. An IgG1 murine monoclonal antibody specific for CD37, MB-1, was labeled with ¹³¹I and tested in a clinical trial in the treatment of NHL (see Press et al., J. Clin. Oncol. 7:1027 (1989); Bernstein et al., Cancer Res. (Suppl.) 50:1017 (1990); Press et al., Front. Radiat. Ther. Oncol. 24:204 (1990); Press et al., Adv. Exp. Med. Biol. 303:91 (1991) and Brown et al., Nucl. Med. Biol. 24:657 (1997)). The MB-1 antibody lacks Fc effector functions, such as antibody-dependent cellular cytotoxicity (ADCC), and the naked MB-1 antibody did not inhibit tumor growth in an in vivo xenograft model (Buchsbaum et al., Cancer Res. 52:6476 (1992)). In addition, an immunoconjugate having adriamycin linked to G28-1, another murine monoclonal anti-CD37, was administered to mice and shown to be internalized with adriamycin being released intracellularly (see, Braslawsky et al., Cancer Immunol. Immunother. 33:367 (1991)). An engineered fusion protein, termed a small modular immunopharmaceutical (SMIP™) product, directed to CD37 is currently being tested in humans (see, e.g., US Patent Application Publications 2003/0133939 and 2007/0059306; PCT Publication No. WO 2009/126944).

Although there has been extensive research carried out on antibody-based therapies, there remains a need in the art for alternative or improved compositions and methods for treating B-cell associated disorders or diseases.

### BRIEF SUMMARY

The present disclosure provides methods, compositions and kits for the combined use of CD37-specific binding molecules and mTOR or PI3K inhibitors to reduce B-cells or treat a disease or disorder associated with aberrant B-cell activity.

In one aspect, the present disclosure provides a method of reducing the number of B-cells or treating a disease or disorder associated with aberrant B-cell activity in a subject having or suspected having the disease or disorder, comprising treating (i.e., administering to) a subject with a therapeutically effective amount of a CD37-specific binding molecule and a therapeutically effective amount of an mTOR or PI3K inhibitor. Additional methods are provided according to claims 2 to 20 and described herein.

In another aspect, the present disclosure provides a kit for treating a non-Hodgkins lymphoma comprising: (a) a unit dosage of a CD37-specific binding molecule, and (b) a unit dosage of an mTOR or PI3K inhibitor. Additional kits are provided according to claim 22 and described herein.

In another aspect, the present disclosure provides a composition, comprising: (a) a CD37-specific binding molecule, and (b) an mTOR or phosphatidylinositol 3-kinase (PI3K) inhibitor. Additional compositions are provided according to claims 24-36 and described herein.

### BRIEF DESCRIPTION OF THE DRAWINGS

FIG. 1 shows the combination effect of CAS-024 and rapamycin on growth of Rec-1 cells. Both molecules were used at equivalent concentrations.

FIG. 2 shows the combination effect of CAS-024 and rapamycin on growth of SU-DHL-6 cells. Both molecules were used at equivalent concentrations.

FIGS. 3A and 3B show Combination Index (CI) plots for the Rec-1 and SU-DHL-6 cell lines. The CI values illustrate the interaction of CAS-024 and rapamycin plotted across (A) effect levels and (B) the mean CI±95% confidence interval for the entire effect range.

FIG. 4 shows the combination effect of CAS-024 and temsirolimus on growth of SU-DHL-6 cells. Both molecules were used at equivalent concentrations.

FIG. 5 shows the combination effect of CAS-024 and temsirolimus on growth of Rec-1 cells. Both molecules were used at equivalent concentrations.

FIG. 6 shows CI plots of the combination of CAS-024 with temsirolimus for the SU-DHL-6 cell line across effect levels.

FIG. 7 shows CI plots of the combination of CAS-024 with temsirolimus for the Rec-1 cell line across effect levels.

FIG. 8 shows CI plots of the combination of CAS-024 with temsirolimus for the Rec-1 and SU-DHL-6 cell lines. The CI values represent the mean CI±95% confidence interval for the entire effect range.

FIG. 9 is a CI plot for CAS-024 with LY294002 for the SU-DHL-6 cell line across effect levels. The values are the mean of three independent experiments.

### DETAILED DESCRIPTION

The present disclosure provides compositions and methods for the combined use of CD37-specific binding molecules and mTOR or PI3K inhibitors to reduce B-cells that were associated with certain diseases or disorders, such as cancer. A surprising result of this combination is that these compounds act synergistically, which results in an increased B-cell reduction. In a related aspect, this disclosure provides methods for treating an individual having or suspected of having a disease associated with aberrant B-cell activity, such as a B-cell lymphoma such as B-cell non-Hodgkins lymphoma (NHL) or a B-cell leukemia such as chronic lymphocytic leukemia, or the like.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “comprise” are used synonymously. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

A “binding domain” or “binding region” according to the present disclosure may be, for example, any protein, polypeptide, oligopeptide, or peptide that possesses the ability to specifically recognize and bind to a biological molecule (e.g., CD37) or complex of more than one of the same or different molecule or assembly or aggregate. Exemplary binding domains include single chain antibody variable regions (e.g., domain antibodies, sFv, scFv, Fab). A variety of assays are known for identifying binding domains of the present disclosure that specifically bind a particular target, including Western blot, ELISA, or Biacore® analysis.

Binding domains and fusion proteins thereof of this disclosure can be capable of binding to a desired degree, including “specifically or selectively binding” a target while not significantly binding other components present in a test sample, if they bind a target molecule with an affinity or Kₐ (i.e., an equilibrium association constant of a particular binding interaction with units of 1/M) of, for example, greater than or equal to about 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. “Low affinity” binding domains refers to those binding domains with a Kₐ of up to 10⁷ M⁻¹, up to 10⁶ M⁻¹, up to 10⁵ M⁻¹, or less. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

The term “CD37-specific binding molecule” refers to a protein, polypeptide, oligopeptide or peptide that preferentially binds to human CD37 protein antigen (see, e.g., GenBank Accession Nos. EAW52467.1, EAW52468.1, BAG62633.1, BAH14719.1, BAG62877.1, NP 001765.1 and NP 001035120.1) over other proteins and binds with a Ka of at least about 10⁶ M⁻¹ (e.g., at least about 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹).

The term “CD37-specific binding domain” refers to a portion or a domain of a CD37-specific binding molecule directly responsible for binding CD37.

A CD37-specific binding domain itself (i.e., without any other portion of the CD37-specific binding molecule) binds to CD37 with a Ka of at least about 10⁶ M⁻¹ (e.g., at least about 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹). A CD37-specific binding domain itself may be sufficient as a CD37-specific binding molecule. Exemplary CD37-specific binding domains include CD37-specific scFv and Fab fragments, which can be based on anti-CD37 antibody variable domains or CDRs, such as variable domains or CDRs from monoclonal antibodies G28-1, IPO24, WR17, MB371, HH1, or HD28.

Terms understood by those in the art of antibody technology are each given the meaning acquired in the art, unless expressly defined differently herein. Antibodies are known to have antigen-binding variable domains, a hinge region, and constant regions that mediate effector function. The term “antibody” refers to an intact antibody comprising at least two heavy (H) chains and two light (L) chains inter-connected by disulfide bonds, as well as an antigen-binding portion of an intact antibody that has or retains the capacity to bind a target molecule. A monoclonal antibody or antigen-binding portion thereof may be non-human, chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

For example, the terms “VL” and “VH” refer to the variable binding domain from an antibody light and heavy chain, respectively. The variable binding domains are made up of discrete, well-defined sub-regions known as “complementarity determining regions” (CDRs) and “framework regions” (FRs). More specifically, each VH and VL domain of an antibody is composed of three CDRs and four FRs, arranged from amino-terminus to carboxyl-terminus in the following order: FR1, CDR1, FR2, CDR2, FR3, CDR3, FR4.

The heavy and light chain variable domains can be fused together through a linker amino acid sequence to form a “single chain variable fragment” (scFv). A “variable domain linker” is an amino acid sequence of about 5 to about 35 amino acids (e.g., (GlyₙSer)ₘ, wherein n and m are integers independently selected from 1 to 6, preferably n is 4 and m is 3, 4, or 5) interposed between and connecting a heavy chain variable domain with a light chain variable domain or connecting a light chain variable domain with a heavy chain variable domain, which provides a spacer function compatible with interaction of the two variable domains so that the resulting polypeptide retains a specific binding affinity to the same target molecule as an antibody having the same light and heavy chain variable regions.

Antibodies have a hinge sequence that is typically situated between the Fab portion and constant region (but a lower section of the hinge may include an amino-terminal portion of the constant region). By way of background, an immunoglobulin hinge acts as a flexible spacer to allow the Fab portion to move freely in space. According to crystallographic studies, an IgG hinge domain can be functionally and structurally subdivided into three regions: the upper, the core or middle, and the lower hinge regions (Shin et al. (1992) Immunol. Rev. 130:87). Exemplary upper hinge regions include EPKSCDKTHT (SEQ ID NO:263) as found in IgG1, ERKCCVE (SEQ ID NO:270) as found in IgG2, ELKTPLGDTT HT (SEQ ID NO:271) or EPKSCDTPPP (SEQ ID NO:272) as found in IgG3, and ESKYGPP (SEQ ID NO:273) as found in IgG4. Exemplary middle or core hinge regions include CPPCP (SEQ ID NO:274) as found in IgG1 and IgG2, CPRCP (SEQ ID NO:275) as found in IgG3, and CPSCP (SEQ ID NO:276) as found in IgG4. While IgG1, IgG2, and IgG4 antibodies each appear to have a single upper and middle hinge, IgG3 has four in tandem—one being ELKTPLGDTTHTCPRCP (SEQ ID NO:277) and three being EPKSCDTPPPCPRCP (SEQ ID NO:278).

IgA and IgD antibodies appear to lack an IgG-like core region, and IgD appears to have two upper hinge regions in tandem (see, for example, ESPKAQASSVPTAQPQAEGSLAKATTAPATTRNT, SEQ ID NO:279 and GRGGEEKKKEKEKEEQEERETKTP, SEQ ID NO:280). Exemplary wild type upper hinge regions found in IgA1 and IgA2 antibodies are VPSTPPTPSPSTPPTPSPS (SEQ ID NO:281) and VPPPPP (SEQ ID NO:282), respectively.

IgE and IgM antibodies, in contrast, lack a typical hinge region and instead have a CH2 domain with hinge-like properties. Exemplary wild-type CH2 upper hinge-like sequences of IgE and IgM are set forth in SEQ ID NO:283 (VCSRDFTPPTVKILQSSSDGGGHFPPTIQLLCLVSGYTPGTINITWLEDG QVMDVDLSTASTTQEGELASTQSELTLSQKHWLSDRTYTCQVTYQGHTFE DSTKKCA) and SEQ ID NO:284 (VIAELPPKVSVFVPPRDGFFGNPRKSKLIC QATGFSPRQIQVSWLREGKQVGSGVTTDQVQAEAKESGPTTYKVTSTLTI KESDWLGQSMFTCRVDHRGLTFQQNASSMCVP), respectively.

As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

As used herein, an “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (preferably having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as set forth in SEQ ID NOS:274-276.

In addition, antibodies contain constant regions. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype, into CH1, CH2, and CH3 (IgA, IgD, IgG), or CH1, CH2, CH3, and CH4 domains (IgE, IgM). A portion of the constant region domains makes up the Fc region (the “fragment crystallizable” region) from an antibody and is responsible for effector functions (such as antibody-dependent cell-mediated cytotoxicity (ADCC), antibody-dependent cellular phagocytosis (ADCP), complement-dependent cytotoxicity (CDC) and complement fixation), binding to Fc receptors (e.g., CD16, CD32, FcRn), long half-life in vivo, protein A binding, and perhaps even placental transfer (see Capon et al. (1989) Nature 337:525).

Exemplary wild type human CH2 domains are set forth in SEQ ID NOS:285-293, wild type human CH3 domains are set forth in SEQ ID NOS:294-302, and wild type human CH4 domains are set forth in SEQ ID NO:303 and 304. An “altered immunoglobulin constant region” refers to an immunoglobulin constant region with a sequence identity to a wild type constant region of at least 75% (e.g., 80%, 82%, 84%, 86%, 88%, 90%, 91%, 92%, 93%, 94%, 95%, 96%, 97%, 98%, 99%, or 99.5%). For example, an “altered immunoglobulin CH2 region” or “altered CH2 region” refers to a CH2 region with a sequence identity to a wild type immunoglobulin CH2 region (e.g., a human CH2) of at least 75% (e.g., 80%, 82%, 84%, 86%, 88%, 90%, 91%, 92%, 93%, 94%, 95%, 96%, 97%, 98%, 99%, or 99.5%). Similarly, an “altered immunoglobulin CH3 region” or “altered CH3 region” refers to a CH3 region with a sequence identity to a wild type immunoglobulin CH3 region (e.g., a human CH3) of at least 75% (e.g., 80%, 82%, 84%, 86%, 88%, 90%, 91%, 92%, 93%, 94%, 95%, 96%, 97%, 98%, 99%, or 99.5%).

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values are generated by the NCBI BLAST2.0 software as defined by Altschul et al. (1997) “Gapped BLAST and PSI-BLAST: a new generation of protein database search programs,” Nucleic Acids Res. 25:3389-3402, with the parameters set to default values.

In certain embodiments, an altered immunoglobulin region or domain only contains conservative amino acid substitutions of a wild type immunoglobulin domain. In certain other embodiments, an altered immunoglobulin domain only contains non-conservative amino acid substitutions of a wild type immunoglobulin domain. In yet other embodiments, an altered immunoglobulin domain contains both conservative and non-conservative amino acid substitutions.

A “conservative substitution” is recognized in the art as a substitution of one amino acid for another amino acid that has similar properties. Exemplary conservative substitutions are well known in the art (see, e.g., PCT Publication No. WO 97/09433, page 10; Lehninger, Biochemistry, Second Edition; Worth Publishers, Inc. NY:NY (1975), pp. 71-77; Lewin, Genes IV, Oxford University Press, NY and Cell Press, Cambridge, Mass. (1990), p. 8). In certain embodiments, a conservative substitution includes a leucine to serine substitution.

“Derivative” as used herein refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.”

A “small modular immunopharmaceutical (SMIP™) protein or polypeptide” refers to a single chain fusion protein that comprises from its amino to carboxy terminus: (i) a binding domain that specifically binds a target molecule, (ii) a linker polypeptide (e.g., an immunoglobulin hinge or derivative thereof), and (iii) (a) an immunoglobulin CH2 polypeptide and an immunoglobulin CH3 polypeptide of IgG, IgA or IgD, or (b) an immunoglobulin CH3 polypeptide and an immunoglobulin CH4 polypeptide of IgM or IgE (see, U.S. Patent Publication Nos. 2003/0133939, 2003/0118592, and 2005/0136049; and PCT Publication No. WO 2005/017148).

A “PIMS protein” is a reverse SMIP molecule wherein the binding domain is disposed at the carboxy-terminus of the fusion protein. Constructs and methods for making PIMS proteins are described in PCT Publication No. WO 2009/023386 and U.S. Patent Application Publication No. US 2009/0148447, which constructs that can contain a CD37 binding domain are incorporated herein by reference. An exemplary PIMS molecule is a single-chain polypeptide comprising, in amino-terminal to carboxy-terminal orientation, a constant sub-region derived from an antibody (e.g., a region that comprises a CH2 domain and a CH3 domain), a linker peptide (e.g., a CD molecule stalk region or a functional variant thereof), and a binding domain (e.g., CD37). In certain embodiments, a PIMS further comprises a second linker peptide disposed amino-terminal to the constant sub-region (e.g., an immunoglobulin hinge region), which may be the same as or different from the linker peptide between the constant sub-region and the binding domain.

A “SCORPION protein” is a fusion protein comprising two binding domains that comprise variable regions from immunoglobulin or immunoglobulin-like molecules. Constructs and methods for making SCORPION proteins are described in PCT Publication No. WO 2007/146968 and U.S. Patent Application Publication No. US 2009/0175867, which constructs that can contain a CD37 binding domain are incorporated herein by reference. An exemplary SCORPION protein is a single chain multivalent or multi-specific binding protein with an effector function, comprising from amino-terminus to carboxy-terminus: (a) a first binding domain comprising variable regions from an immunoglobulin or immunoglobulin-like molecule, (b) a first linker peptide, (c) an immunoglobulin constant sub-region providing an effector function, (d) a second linker peptide, and (e) a second binding domain comprising variable regions from an immunoglobulin or immunoglobulin-like molecule. In certain embodiments, the first and second binding domains bind the same target (e.g., CD37). In certain other embodiments, the first and second binding domains bind different targets.

As used herein, unless otherwise provided, a position of an amino acid residue in a variable region of an immunoglobulin molecule or a fusion protein containing immunoglobulin regions or domains is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94; Kabat, supra).

“B-cell associated disorder or disease” or “a disease or disorder associated with aberrant B-cell activity” refers to a disease or disorder associated with (e.g., causing or resulting from) aberrant B-cell activity or activity that deviates from the normal, proper, or expected course. For example, a B-cell associated disorder or disease may include inappropriate proliferation of B-cells that have damaged or defective DNA or other cellular components. Aberrant B-cell activity may include cell proliferation characterized by inappropriately high levels of B-cell division, inappropriately low levels of B-cell apoptosis, or both. Such diseases may have, for example, single or multiple local abnormal proliferations of B-cells, groups of B-cells or tissue(s), whether cancerous or non-cancerous, benign or malignant. A B-cell associated disorder or disease may also include aberrant antibody production, such as production of autoantibodies, or overproduction of antibodies more desirable when produced at normal levels. It is also contemplated herein that aberrant B-cell activity may occur in certain subpopulations of B-cells and not in other subpopulations, or may include inappropriate stimulation of T-cells, such as by inappropriate antigen presentation to T-cells or by other B-cells pathway.

“Treatment” or “treating” refers to either a therapeutic treatment or prophylactic/preventative treatment. A therapeutic treatment may improve at least one symptom of disease in an individual receiving treatment or may delay worsening of a progressive disease in an individual, or prevent onset of additional associated symptoms or diseases, or any combination thereof.

A “therapeutically effective amount (or dose)” or “effective amount (or dose)” of a specific binding molecule (e.g., a CD37-specific binding molecule) or compound (e.g., an mTOR inhibitor, PI3K inhibitor) refers to that amount of the compound or combination of compounds sufficient to result in amelioration of one or more symptoms of the disease being treated, delaying worsening of a progressive disease, or preventing onset of additional associated symptoms or diseases, or any combination thereof.

“A subject having, or suspected of having, a disease associated with aberrant B-cell activity” is a subject (human or another animal) in whom a disease or a symptom of a disorder may be caused by aberrant B-cell activity or B-cell proliferation, may be exacerbated by aberrant B-cell activity, or may be relieved by regulation of B-cell activity. Examples of such diseases include a B-cell malignancy or B-cell cancer (e.g., B-cell lymphoma, B-cell leukemia or B-cell myeloma), a disease characterized by autoantibody production (e.g., autoimmune diseases) or inflammation or a disease characterized by inappropriate T-cell stimulation caused by inappropriate B-cell antigen presentation to T-cells or caused by other pathways involving B-cells.

### CD37-Specific Binding Molecules

CD37-specific binding molecules useful for the combination therapy described herein contain a CD37-specific binding domain. A CD37-specific binding domain may be used alone or in a scaffold, including in the form of an anti-CD37 antibody or an antigen binding fragment thereof, an anti-CD37 antibody Fab portion or (Fab)₂ portion, an anti-CD37 single chain Fv (scFv), an anti-CD37 SMIP protein, an anti-CD37 PIMS protein, an anti-CD37 SCORPION protein, or the like.

Immunoglobulin-based CD37-specific binding domains useful in the instant invention include those known in the art as described herein, or those generated by a variety of methods known in the art (see, e.g., U.S. Pat. Nos. 6,291,161 and 6,291,158). For example, CD37-specific binding domains may be identified by screening a Fab phage library for Fab fragments that specifically bind to CD37 (see Hoet et al. (2005) Nature Biotechnol. 23:344). Additionally, traditional strategies for hybridoma development, such as using CD37 as an immunogen in convenient systems (e.g., mice, HuMAb Mouse®, TC Mouse™, KM-Mouse®, llamas, sheep, chicken, rats, hamsters, rabbits, etc.), can be used to develop anti-CD37 antibodies having CD37-specific binding domains of interest.

Sources of further binding domains include CD37-specific antibody variable domains from various species (which can be formatted as antibodies, sFvs, scFvs, Fabs, or soluble VH domain or domain antibodies), including human, rodent, avian, and ovine. Additional sources of binding domains include variable domains of antibodies from other species, such as camelid (from camels, dromedaries, or llamas (Ghahroudi et al. (1997) FEBS Letters 414:521; Vincke et al. (2009) J. Biol. Chem. 284:3273; and Hamers-Casterman et al. (1993) Nature, 363:446; and Nguyen et al. (1998) J. Mol. Biol., 275:413), nurse sharks (Roux et al. (1998) Proc. Nat'l. Acad. Sci. (USA) 95:11804), spotted ratfish (Nguyen et al. (2002) Immunogenetics, 54:39), or lamprey (Herrin et al., (2008) Proc. Nat'l. Acad. Sci. (USA) 105:2040 and Alder et al. (2008) Nature Immunol. 9:319). These antibodies can apparently form antigen-binding regions using only heavy chain variable region, i.e., these functional antibodies are homodimers of heavy chains only (referred to as “heavy chain antibodies”) (Jespers et al. (2004) Nature Biotechnol. 22:1161; Cortez-Retamozo et al. (2004) Cancer Res. 64:2853; Baral et al. (2006) Nature Med. 12:580, and Barthelemy et al. (2008) J. Biol. Chem. 283:3639).

Other alternative sources of CD37-specific binding domains includes sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), ankyrin repeat proteins (Binz et al. (2003) J. Mol. Biol. 332:489 and Binz et al. (2004) Nature Biotechnology 22:575), fibronectin binding domains (Richards et al. (2003) J. Mol. Biol. 326:1475; Parker et al. (2005) Protein Eng. Des. Sel. 18:435 and Hackel et al. (2008) J. Mol. Biol. 381:1238), cysteine-knot miniproteins (Vita et al. (1995) Proc. Nat'l. Acad. Sci. (USA) 92:6404; Martin et al. (2002) Nature Biotechnol. 21:71 and Huang et al. (2005) Structure 13:755), tetratricopeptide repeat domains (Main et al. (2003) Structure 11:497 and Cortajarena et al. (2008) ACS Chem. Biol. 3:161), leucine-rich repeat domains (Stumpp et al. (2003) J. Mol. Biol. 332:471), lipocalin domains (see, e.g., PCT Publication No. WO 2006/095164, Beste et al. (1999) Proc. Nat'l. Acad. Sci. (USA) 96:1898 and Schönfeld et al. (2009) Proc. Nat'l. Acad. Sci. (USA) 106:8198), V-like domains (see, e.g., US Patent Application Publication No. 2007/0065431), C-type lectin domains (Zelensky and Gready (2005) FEBS J. 272:6179; Beavil et al. (1992) Proc. Nat'l. Acad. Sci. (USA) 89:753 and Sato et al. (2003) Proc. Nat'l. Acad. Sci. (USA) 100:7779), mAb² or Fcab™ (see, e.g., PCT Publication Nos. WO 2007/098934; WO 2006/072620), or the like (Nord et al. (1995) Protein Eng. 8:601; Nord et al. (1997) Nature Biotechnol. 15:772; Nord et al. (2001) Eur. J. Biochem. 268:4269; and Binz et al. (2005) Nature Biotechnol. 23:1257).

In certain embodiments, a CD37-specific binding domain contains a VH domain derived from or based on a VH of an anti-CD37 monoclonal antibody. In further embodiments, a CD37-specific binding domain contains a VL domain derived from or based on a VL of an anti-CD37 monoclonal antibody. In still further embodiments, a CD37-specific binding domain contains a VH domain and a VL domain derived from or based on a VH and VL, respectively, from a single anti-CD37 monoclonal antibody or from at least two different anti-CD37 monoclonal antibodies. In a preferred embodiment, the VH and VL domains are from monoclonal antibody G28-1 (SEQ ID NOS:241 and 236, respectively) or from monoclonal antibody or SMIP protein CAS-024 (SEQ ID NOS:245 and 238, respectively).

In certain embodiments, a CD37-specific binding domain contains VH and VL domains that are each independently modified to contain one or more (e.g., 2, 3, 4, 5, 6, 7, 8, 9, 10) amino acid insertions, one or more (e.g., 2, 3, 4, 5, 6, 7, 8, 9, 10) amino acid deletions, one or more (e.g., 2, 3, 4, 5, 6, 7, 8, 9, 10) amino acid substitutions (e.g., conservative amino acid substitutions), or a combination thereof, when compared with the wild type VH and VL domains, respectively, of the parent anti-CD37 monoclonal antibody or antibodies. The insertion(s), deletion(s) or substitution(s) may be anywhere in the VH domain, VL domain or both, including at the amino- or carboxy-terminus or both ends of each or both domains, provided that each CDR comprises zero changes or at most one, two, or three changes and provided that a CD37 binding domain containing the modified VH domain, VL domain, or both can specifically bind CD37 with an affinity similar to or greater than the wild type binding domain.

CD37-specific binding domains comprising immunoglobulin VL and VH domains will comprise a total of two, three, four, five, or preferably six CDRs (i.e., three in VL and three in VH). Such CDRs may be human or non-human CDRs, or variants thereof comprising at most one, two, or three amino acid changes per CDR. In certain embodiments, a CD37-specific binding domain comprises (a) a light chain variable domain that comprises a light chain CDR1, a light chain CDR2, and a light chain CDR3, and (b) a heavy chain variable domain that comprises a heavy chain CDR1, a heavy chain CDR2, and a heavy chain CDR3.

Exemplary CDRs include CDR1 of the light chain as set forth in SEQ ID NO:61 (RASENVYSYLA), SEQ ID NO:62 (RTSENVYSYLA), SEQ ID NO:311 (KASQDVSTAVA), or SEQ ID NO:312 (RASSSIVYMH); CDR1 of the heavy chain as set forth in SEQ ID NO:63 (GYNMN), SEQ ID NO:313 (GYSFTDFNMY), or SEQ ID NO:314 (GFTFRSYGMS); CDR2 of the light chain as set forth in SEQ ID NO:64 (FAKTLAE), SEQ ID NO:315 (WASTRHT), or SEQ ID NO:316 (DTSKLAS); CDR2 of the heavy chain as set forth in SEQ ID NO:65 (NIDPYYGGTTYNRKFKG), SEQ ID NO:317 (YIDPYNGDTTYNQKFKG), or SEQ ID NO:318 (SINSDGGSTYYPDVKG); CDR3 of the light chain as set forth in SEQ ID NO:66 (QHHSDNPWT), SEQ ID NO:319 (QQHYSTPLT), or SEQ ID NO:320 (HQRSSYPTT); and CDR3 of the heavy chain as set forth in SEQ ID NO:67 (SVGPFDY), SEQ ID NO:68 (SVGPFDS), SEQ ID NO:69 (SVGPMDY), SEQ ID NO:321 (GPNWVAMDY), or SEQ ID NO:322 (GGALIVTSDAMDY). Preferred light chain CDR1 is SEQ ID NO:61 (RASENVYSYLA) and preferred heavy chain CDR3 include SEQ ID NO:68 (SVGPFDS) or SEQ ID NO:69 (SVGPMDY). Additional exemplary CDRs are set forth in SEQ ID NOS:128-137 (for light chain CDR1 sequences), 138 and 139 (for heavy chain CDR2 sequences), and 213 and 215-219 (for heavy chain CDR3 sequences). Further exemplary CDRs may be found in PCT Publication No. WO 2009/126944, which CDRs are incorporated herein by reference.

In further embodiments, binding domains specific for human CD37 comprise immunoglobulin VL and VH domains that are non-human, humanized, or human. As used herein, “humanized CD37-specific binding domain” refers to a binding domain comprising non-human immunoglobulin VL and VH domains that form a binding domain specific for human CD37 and each have at least one, two, three, or preferably four human framework regions.

A “human framework region” refers to human framework regions (FRs) found in immunoglobulin variable domains, which may be (i) wild type human FRs from naturally occurring germ line or somatic sequences, (ii) altered human FRs with less than about 50% (e.g., preferably less than about 45%, 40%, 30%, 25%, 20%, 15%, 10%, 5%, or 1%) of the amino acids corresponding to non-human amino acids at the corresponding FR positions, or (iii) altered non-human FRs with at least about 50% (e.g., at least about 55%, 60%, 65%, 70%, 75%, 80%, 85%, 90%, 95%, 96%, 97%, 98%, or 99%) of the amino acids corresponding to human amino acids at the corresponding FR positions so that immunogenicity is reduced.

Exemplary human FRs are set forth in SEQ ID NOS:140-146 (human heavy chain FR1), SEQ ID NOS:147, 150 and 151 (human heavy chain FR2), SEQ ID NO:154-160 (human heavy chain FR3), SEQ ID NOS: 161-163, 168 and 169 (human heavy chain FR4), SEQ ID NOS:170-172, 175, and 177-181 (human light chain FR1), SEQ ID NOS:182, 184-188 and 191 (human light chain FR2), SEQ ID NOS:194-198, 203 and 205 (human light chain FR3), and SEQ ID NOS:206-210 (human light chain FR4). Additional exemplary human FR regions may be found in the CD37-specific SMIP proteins provided herein, such as in CAS-001, CAS-002, CAS-003, and CAS-024 (SEQ ID NOS:248, 249, 250 and 253, respectively).

In certain embodiments, CD37-specific binding domains comprise a humanized heavy chain variable region that comprises from its amino terminus to carboxyl terminus: human heavy chain FR1, heavy chain CDR1 as set forth in SEQ ID NO:63, human heavy chain FR2, heavy chain CDR2 as set forth in SEQ ID NO:65, human heavy chain FR3, heavy chain CDR3 as set forth in SEQ ID NO:67, 68 or 69, and human heavy chain FR4. In further embodiments, CD37-specific binding domains comprise consist essentially of, or consist of a humanized heavy chain variable region that comprises from its amino terminus to carboxyl terminus: human heavy chain FR1 as set forth in SEQ ID NO:144, heavy chain CDR1 as set forth in SEQ ID NO:63, human heavy chain FR2 as set forth in SEQ ID NO:151, heavy chain CDR2 as set forth in SEQ ID NO:65, human heavy chain FR3 as set forth in SEQ ID NO:158, heavy chain CDR3 as set forth in SEQ ID NO:67, 68 or 69, and human heavy chain FR4 as set forth in SEQ ID NO:161. Additional exemplary humanized light chains are set forth in SEQ ID NOS:242-245 and include the light chains in humanized CD37-specific SMIP proteins provided herein.

In still further embodiments, only the light or heavy chain variable domain is humanized. For example, CD37-specific binding domains may comprise a humanized light chain variable domain (i.e., a light chain variable region that comprises at least one human FR) and a nonhuman heavy chain variable chain region (e.g., mouse or rat). Alternatively, CD37-specific binding domains may comprise a non-human light chain variable domain (e.g., mouse or rat) and a humanized heavy chain variable chain domain (i.e., a heavy chain variable region that comprises at least one human FR). Both types of CD37-specific binding domains may be referred to as a “hybrid human-nonhuman CD37-specific binding domain” or as a “chimeric CD37-specific binding domain.”

In certain embodiments, CD37-specific binding domains are in the form of a scFv fragment. In a preferred embodiment, the CD37-specific binding domain is a human or humanized CD37-specific scFv that comprises a light chain variable domain and a heavy chain variable domain joined together via a variable domain linker. In further embodiments, both the light and heavy chain variable domains are humanized, and may comprise both a humanized light chain variable domain as set forth in SEQ ID NO:238 and a humanized heavy chain variable domain as set forth in SEQ ID NO:245. In still further embodiments, only the light or heavy chain variable domain of the scFv is humanized.

In a preferred embodiment, the carboxyl terminus of the VL domain in a humanized CD37-specific scFv is linked to the amino terminus of the VH domain via a variable domain linker. Thus, the resulting scFv has from its amino terminus to its carboxyl terminus: the VL domain, the variable domain linker, and the VH domain. In another preferred embodiment, the carboxyl terminus of the VH domain in a humanized CD37-specific scFv is linked to the amino terminus of the VL domain via a variable domain linker. Thus, the resulting scFv has from its amino terminus to its carboxyl terminus: the VH domain, the variable domain linker, and the VL domain. In a preferred embodiment, the VH and VL domains of an scFv are from monoclonal antibody G28-1 (SEQ ID NOS:241 and 236, respectively) or from monoclonal antibody or SMIP protein CAS-024 (SEQ ID NOS:245 and 238, respectively), and the variable domain linker has about five to about 35 amino acids, preferably about 15 to about 25 amino acids.

In certain embodiments, a variable domain linker joining the VH and VL domains or the VL and VH domains are those belonging to the (GlyₙSer) family as described herein. For example, the variable domain linker comprises (GlyₙSer)ₘ, wherein n and m may be an integer independently selected from 1 to 6. In certain preferred embodiments, n is 4 and m is 1, 2, 3, 4, 5, or 6, and more preferably n is 4 and m is 3, 4, or 5. In further embodiments, one or two amino acids other than Gly or Ser may be present at the amino terminus, carboxyl terminus or both termini. In certain other embodiments, one or two amino acids of the (GlyₙSer)ₘ can be substituted with an amino acid other than Gly or Ser. An exemplary variable domain linking sequence having the sequence (Gly₄Ser)₅ is set forth in SEQ ID NO:229. Additional exemplary variable domain linking sequences are set forth in SEQ ID NOS:225-228.

In certain embodiments, CD37-specific binding molecule or binding domain competes for binding to a human CD37 protein with a G28-1 monoclonal antibody (mAb), a CAS-024 mAb, or a CAS-024 SMIP protein. As used herein, “competes with binding” means that binding to a target molecule by a binding molecule specific for that target is reduced or inhibited by the presence of another binding molecule specific for the same target—meaning the two different binding molecules, such as two different anti-CD37 antibodies, may bind to the same or similar antigen binding site or epitope (e.g., sequential or conformational), or may sterically hinder binding to neighboring antigen binding sites or epitopes. For example, G28-1 mAb binding to CD37 is reduced in the presence of CAS-024 SMIP protein when compared to the binding of CD37 by G28-1 mAb in the absence of CAS-024 SMIP protein (i.e., CAS-024 is competing with G28-1 mAb for binding to CD37). Competitive binding assays are known in the art, such as those described in Example 2 of PCT Publication No. WO 2007/014278 and Examples 4-6 of PCT Publication No. WO 2009/126944, and may be used to determine whether a given CD37-specific binding domain or CD37-specific binding molecule is capable of competing with a G28-1 mAb, a CAS-024 mAb, or a CAS-024 SMIP protein for binding to CD37.

CD37-specific binding molecules of the present disclosure may comprise a hinge or linker polypeptide that joins a CD37-specific binding domain to an immunoglobulin constant of Fc region. As used herein, a “hinge region,” a “hinge,” a “hinge polypeptide,” or a “linker polypeptide” refers to (a) a wild type immunoglobulin hinge region; (b) an altered immunoglobulin hinge region; (c) a peptide based on or derived from an interdomain region of an immunoglobulin superfamily member; (d) a cluster of differentiation (CD) molecule stalk region or a functional variant thereof; or (e) a stalk region of C-type lectins, a family of type II membrane proteins (see, e.g., exemplary lectin stalk region sequences set forth in of PCT Application Publication No. WO 2007/146968, such as SEQ ID NOS:111, 113, 115, 117, 119, 121, 123, 125, 127, 129, 131, 133, 135, 149, 151, 153, 155, 157, 159, 161, 163, 165, 167, 169, 231, 233, 235, 237, 239, 241, 243, 245, 247, 249, 251, 253, 255, 257, 259, 261, 263, 265, 267, 269, 271, 273, 275, 277, 279, 281, 287, 289, 297, 305, 307, 309-311, 313-331, 346, 373-377, 380, or 381 from that publication, which sequences are incorporated herein by reference), or a functional variant thereof.

In certain embodiments, a hinge region is a wild type immunoglobulin hinge region, such as an IgG hinge, IgA hinge, IgD hinge, IgE hinge or a functional fragment thereof (e.g., 4 to 20 or 5 to 15 amino acids in length) that comprises at least an IgG1 core hinge region. In certain preferred embodiments, a hinge region may be an antibody hinge region selected from human IgG1, human IgG2, human IgG3, human IgG4, or functional variants thereof. In some embodiments, the hinge region is a wild type human immunoglobulin hinge region or functional variant thereof. Exemplary hinges for such embodiments are wild type human IgG1 hinge region as set forth in SEQ ID NO:90, wild type human IgA1 hinge as set forth in SEQ ID NO:115, wild type human IgA2 hinge as set forth in SEQ ID NO:116, wild type human IgG3 hinge as set forth in SEQ ID NO:118, a portion of human IgG3 hinge as set forth in SEQ ID NO:258, and human IgD hinge as set forth in SEQ ID NO:127. In certain embodiments, one or more amino acid residues may be added at the amino- or carboxy-terminus of a wild type immunoglobulin hinge region as part of fusion protein construct design. Such amino acid residues are referred to as “junction amino acids” (see, e.g., SEQ ID NOS:231-235).

In certain embodiments, the hinge region is an altered (mutated) wild type immunoglobulin hinge region, such as an altered wild type IgG immunoglobulin hinge region. For example, the wild type human IgG1 hinge region contains three cysteine residues—the most N-terminal cysteine is referred to the first cysteine, whereas the most C-terminal cysteine in the hinge region is the third cysteine. In certain embodiments, the mutated human IgG1 hinge region has only two cysteine residues, such as a human IgG1 hinge region with one of the first, second, or third cysteines substituted with a serine, preferably the second cysteine. In certain other embodiments, a mutated human IgG1 hinge region has only one cysteine residue, preferably the third cysteine. In certain embodiments, the proline C-terminal to the third cysteine in the human IgG1 hinge region is substituted, for example, by a serine. Exemplary mutated human IgG1 hinge regions are as set forth in SEQ ID NOS:92, 94, 102, 104, 255, 256, 106, 108, 257, 96, 110, 112, 98, and 100. Exemplary mutated portions of human IgG3 hinge regions are as set forth in SEQ ID NOS:120, 126, 259-261, 122, and 124. In certain embodiments, one or more amino acid residues may be added at the amino- or carboxy-terminus of a mutated immunoglobulin hinge region as part of fusion protein construct design. Examples of such modified hinge regions are indicated in italics in SEQ ID NOS:231-235.

In certain embodiments, a hinge region comprises or has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a wild type immunoglobulin hinge region, such as a wild type human IgG1, IgG2, IgG3, IgG4, IgA1, IgA2, IgD and IgE hinges.

Alternative hinge or linker sequences may be crafted from portions of cell surface receptors that connect IgV-like or IgC-like domains. Regions between IgV-like domains where a cell surface receptor contains multiple IgV-like domains in tandem and between IgC-like domains where a cell surface receptor contains multiple tandem IgC-like regions could also be used as a connecting region or linker peptide. Representative hinge or linker sequences of the interdomain regions between the IgV-like and IgC-like or between the IgC-like or IgV-like domains are found in CD2, CD4, CD22, CD33, CD48, CD58, CD66, CD80, CD86, CD96, CD150, CD166, and CD244. More alternative hinges may be crafted from disulfide-containing regions of Type II receptors from non-immunoglobulin superfamily members, such as CD69, CD72, and CD161.

In certain embodiments, hinge or linker sequences have 2 to 150 amino acid, 5 to 60 amino acids, 2 to 40 amino acids, preferably have 8-20, more preferably have 12-15 amino acids, and may be primarily flexible, but may also provide more rigid characteristics or may contain primarily a helical structure with minimal β sheet structure. Preferably, hinge and linker sequences are stable in plasma and serum and are resistant to proteolytic cleavage. In certain embodiments, the first lysine in the IgG1 upper hinge region is mutated to minimize proteolytic cleavage, preferably the lysine is substituted with methionine, threonine, alanine or glycine, or is deleted. Nucleic acid sequences encoding exemplary linkers are set forth in SEQ ID NOS:89, 91, 93, 95, 97, 99, 101, 103, 105, 107, 109, 111, 117, 119, 121, 123, and 125.

CD37-specific binding molecules of the present disclosure may comprise a constant sub-region derived from an antibody, such as CH2 and CH3 regions of IgG, IgA, or IgD and CH3 and CH4 regions of IgM or IgE.

A CH2 domain that forms a portion of a CD37-specific binding molecule may be a wild type or altered immunoglobulin CH2 domain based on or derived from certain immunoglobulin classes or subclasses (e.g., IgG1, IgG2, IgG3, IgG4, IgA1, IgA2, or IgD) and from various species (including human, mouse, rat, and other mammals). In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS:285, 290-292 and 286-288, respectively. In certain preferred embodiments, the CH2 domain is a wild type human IgG1 CH2 domain as set forth in SEQ ID NO:285. In certain embodiments, a CH2 domain is an altered human immunoglobulin CH2 domain, such as an altered CH2 domain based on or derived from a wild-type CH2 domain of human IgG1, IgG2, IgG3, IgG4, IgA1, IgA2, or IgD antibodies. For example, an altered CH2 domain may be a human IgG1 CH2 domain with one, two, three, four, five, six or more mutations at positions 234-238, 253, 255-258, 290, 297, 310, 318, 320, 322, 331, and 339 (positions are numbered according to EU numbering). In certain embodiments, an altered CH2 domain comprises: (i) an amino acid substitution at the asparagine of position 297; (ii) one or more amino acid substitutions or deletions at positions 234-238; (iii) at least one amino acid substitution or deletion at positions 253, 310, 318, 320, 322, or 331; (iv) an amino acid substitution at the asparagine of position 297 and one or more substitutions or deletions at positions 234-238; (v) an amino acid substitution at the asparagine of position 297 and at least one substitution or deletion at position 253, 310, 318, 320, 322, or 331; (vi) one or more amino acid substitutions or deletions at positions 234-238, and at least one amino acid substitution or deletion at position 253, 310, 318, 320, 322, or 331; or (vi) an amino acid substitution at the asparagine of position 297, one or more amino acid substitutions or deletions at positions 234-238, and at least one amino acid substitution or deletion at position 253, 310, 318, 320, 322, or 331. For example, in certain embodiments, the altered CH2 domain is a human IgG1 CH2 domain with alkaline substitution at position 297. In certain other embodiments, the altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:305). In certain other embodiments, the altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:306). The mutations at the above-noted positions may reduce or eliminate ADCC activity, ADCP activity, Fc receptor binding, or complement fixation.

A CH3 domain that forms a portion of a CD37-specific binding molecule may be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:294, 299-301, 295-298 and 302, respectively. In certain preferred embodiments, the CH3 domain is a wild type human IgG1 CH3 domain as set forth in SEQ ID NO:294. In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain may be a human IgG1 CH3 domain with one or two mutations at positions H433 and N434 (positions are numbered according to EU numbering). The mutations in such positions may be involved in complement fixation. In certain other embodiments, an altered CH3 domain may be a human IgG1 CH3 domain but with one or two amino acid substitutions at position F405 or Y407. The amino acids at such positions are involved in interacting with another CH3 domain.

A CH4 domain that forms a portion of a CD37-specific binding molecule may be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:303 and 304, respectively. In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, a constant sub-region of a CD37-specific binding molecule comprises a combination of CH2, CH3 and/or CH4 domains (i.e., more than one constant sub-domain selected from CH2, CH3 and CH4). For example, a constant sub-region may comprise CH2 and CH3 domains or CH3 and CH4 domains. The multiple constant sub-domains that form a constant sub-region may be based on or derived from the same immunoglobulin molecule (e.g., a constant sub-region formed from human IgG1 CH2 and CH3 as set forth in SEQ ID NO:246), or the same class or subclass immunoglobulin molecules. Alternatively, the multiple constant sub-domains may be based on or derived from different immunoglobulin molecules, or different classes or subclasses immunoglobulin molecules. For example, in certain embodiments, a constant sub-region comprises both human IgM CH3 domain and human IgG1 CH3 domain.

In certain preferred embodiments, a constant sub-region comprises a wild type human IgG1 CH2 domain and a wild type human IgG1 CH3 domain. In certain other preferred embodiments, a constant sub-region comprises an altered human IgG1 CH2 domain (e.g., having an amino acid mutation at N297, having an amino acid mutation at N297 and at least one additional amino acid mutation at positions 234-238, or having amino acid mutations at positions 234, 235, 237, 318, 320 and 322) and a wild type human CH3 domain, so that the constant sub-region does not promote immunological activities, such as ADCC, ADCP, CDC, Fc receptor binding, or any combination thereof. In other embodiments, an altered human IgG1 CH2 domain can have mutations known in the art to enhance immunological activities, such as ADCC, ADCP, CDC, Fc receptor binding, or any combination thereof. In certain other preferred embodiments, a constant sub-region comprises a wild type human IgM CH3 domain and a wild type human IgM CH4 domain, or a wild type human IgE CH3 domain and a wild type human IgE CH4 domain.

In certain embodiments, a CD37-specific binding molecule may contain one or more additional regions. Such additional regions may be a leader sequence at the amino-terminus for secretion of an expressed CD37-specific binding molecule, a tail sequence at its carboxy-terminus for identification or purification purposes (e.g., epitope tags for detection or purification, including a 6-Histidine tag or a FLAG epitope), or additional amino acid residues that arise from use of specific expression systems. Exemplary leader peptides of this disclosure include natural leader sequences or others, such as those as set forth in SEQ ID NOS:223 and 224.

In certain embodiments, fusion proteins may have one or a few (e.g., 2-8) amino acid residues between two domains (such as between immunoglobulin variable domains and a linker polypeptide, between a binding domain and a linker polypeptide or hinge, between a linker polypeptide or hinge and an immunoglobulin CH2 region polypeptide, or between an immunoglobulin CH2 region polypeptide and an immunoglobulin CH3 region polypeptide), such amino acid residues resulting from construct design of the fusion protein (e.g., amino acid residues resulting from the use of a restriction enzyme site during the construction of a nucleic acid molecule encoding a single chain polypeptide). As described herein, such amino acid residues may be referred to as “junction amino acids” or “junction amino acid residues.”

As used herein, a protein “consists essentially of” one domain (e.g., a CD37-specific binding domain) or several domains (e.g., a CD37-specific binding domain, a linker polypeptide, an immunoglobulin CH2 region, and an immunoglobulin CH3 region) if the other portions of the protein (e.g., amino acids at the amino- or carboxy-terminus or between two domains), in combination, contribute to at most 20% (e.g., at most 15%, 10%, 8%, 6%, 5%, 4%, 3%, 2% or 1%) of the length of the protein and do not substantially affect (i.e., do not reduce the activity by more than 50%, such as more than 40%, 30%, 25%, 20%, 15%, 10%, or 5%) protein activity, such as the affinity to CD37 or the ability to reduce the number of B-cells. In certain embodiments, a CD37-specific binding molecule is a SMIP protein consisting essentially of a CD37-specific binding domain, an immunoglobulin hinge polypeptide, an immunoglobulin CH2 region polypeptide, and an immunoglobulin CH3 region polypeptide. Such molecules may further comprise junction amino acids at the amino- or carboxy-terminus of the molecule or between two different domains (e.g., between the binding domain and the hinge polypeptide, between the hinge polypeptide and the immunoglobulin CH2 region polypeptide, and/or between the immunoglobulin CH2 region polypeptide and the immunoglobulin CH3 region polypeptide).

In certain embodiments, CD37-specific binding molecules are anti-CD37 antibodies, including those known in the art. Exemplary anti-CD37 antibodies include HD28, G28-1, HH1, BI14, WR17 and F93G6 used in characterizing the CD37 antigen in the Third HLDA Workshop (See, Ling and MacLennan, pp. 302-335 in Leucocyte Typing III. White Cell Differentiation Antigens, Oxford University Press, 1987). Other CD37-specific antibodies that have been described include RFB-7, Y29/55, MB-1, M-B371, M-B372 and IPO-24 (see Moldenhaurer (2000) J. Biol. Regul. Homeost. Agents 14: 281, finding that all these antibodies recognize a single CD37 epitope). Schwartz-Albiez et al. (J. Immunol. 140:905, 1988) note that the epitope is likely situated in the carbohydrate moiety of CD37. Another CD37-specific antibody is SB3 (Biosys). In certain preferred embodiments, any of these anti-CD37 antibodies are chimeric or humanized antibodies or antigen-binding portions thereof for use in combination with an mTOR inhibitor or a PI3K inhibitor as described herein.

In a preferred embodiment, a CD37 binding domain of this disclosure is an antigen-binding portion of an antibody or includes immunoglobulin variable domains that specifically bind to CD37. Exemplary CD37 antigen-binding portions of an antibody include (i) a fragment antigen-binding (Fab) portion, a monovalent fragment consisting of VL, VH, CL and CH1 domains; (ii) a F(ab′)2 fragment, a bivalent fragment comprising two Fab fragments linked by a disulfide bridge at the hinge region; (iii) an Fd fragment consisting of VH and CH1 domains; (iv) an Fv fragment consisting of VL and VH domains from a single arm of an antibody, (v) a domain Ab fragment (Ward et al. (1989) Nature 341:544) consisting of a VH domain; (vi) a single chain variable fragment (scFv) consisting of VL and VH domains joined by a 5-35 amino acid linker (see, e.g., Huston et al. (1988) Proc. Natl. Acad. Sci. USA 85:5879; Shan et al. (1999) J. Immunol. 162:6589), and (vii) an isolated CDR.

In certain embodiments, CD37-specific binding molecules are CD37-specific SMIP polypeptides. For example, a CD37-specific binding molecule may be a CD37-specific SMIP polypeptide that comprises from its amino to carboxy terminus: (a) a CD37-specific binding domain, (ii) a hinge region or linker polypeptide, (iii) (a) an immunoglobulin CH2 polypeptide of IgG, IgA or IgD and an immunoglobulin CH3 polypeptide of IgG, IgA or IgD, or (b) an immunoglobulin CH3 polypeptide of IgM or IgE and an immunoglobulin CH4 polypeptide of IgM or IgE. The CD37-specific binding domain, the linker polypeptide, the immunoglobulin CH2 polypeptide, the immunoglobulin CH3 polypeptide, the immunoglobulin CH4 polypeptide are as described herein.

Exemplary CD37-specific SMIP polypeptides comprise the sequence set forth in SEQ ID NO:2 or 253. Additional exemplary CD37-specific SMIP polypeptides are described in PCT Publication No. WO 2005/017148, such as (1) G28-1 scFv (SSS—S) H WCH2 WCH3 comprising a G28-1 scFv, an altered human IgG1 hinge in which all three cysteine residues and a proline carboxyl terminus to the third cysteine in a human IgG1 hinge region are mutated to serine residues, and wild type human IgG1 CH2 and CH3 domains; (2) G28-1 scFv IgAH WCH2 WCH3 comprising a G28-1 scFv, a portion of human IgA hinge, and human IgG1 CH2 and CH3 domains; (3) G28-1 scFv VHL11S(SSS—S) H WCH2 CH3 comprising a G28-1 scFv, an altered human IgG1 hinge in which all three cysteine residues and a proline carboxyl terminus to the third cysteine in the hinge region are mutated to serine residues, and human IgG1 CH2 and CH3 domains, wherein the leucine at position 11 of the heavy chain variable region is substituted with a serine; (4) G28-1 scFv VH L11S(CSS—S) H WCH2 CH3 comprising a G28-1 scFv, an altered human IgG1 hinge in which the cysteine residues at the second and third positions and a proline carboxyl terminus to the third cysteine are substituted with serine residues, and human IgG1 CH2 and CH3 domains, wherein the leucine at position 11 of the heavy chain variable region is substituted with a serine; (5) G28-1 scFv VHL11S(CSC—S) H WCH2 CH3 comprising a G28-1 scFv, an altered human IgG1 hinge in which the cysteine residue at the second position and a proline carboxyl terminus to the cysteine at the third position were substituted with serine residues, and human IgG1 CH2 and CH3 domains, wherein the leucine at position 11 of the heavy chain variable region is substituted with a serine; (6) G28-1 scFv VH11S(SSC—P) H WCH2 WCH3 comprising a G28-1 scFv, an altered human IgG1 hinge in which the first and second cysteine residues in the hinge region are mutated to serine residues, and human IgG1 CH2 and CH3 domains, wherein the leucine at position 11 of the heavy chain variable region is substituted with a serine; (7) G28-1 scFv VH11S(SCS—S) H WCH2 WCH3 comprising a G28-1 scFv, an altered human IgG1 hinge in which the first and third cysteine residues and a proline carboxyl terminus to the third cysteine in the hinge regions are mutated to serine residues, and human IgG1 CH2 and CH3 domains, wherein the leucine at position 11 of the heavy chain variable region is substituted with a serine; (8) G28-1 scFv VHL11S(CCS—P) H WCH2 WCH3 comprising a G28-1 scFv, an altered human IgG1 hinge in which the third cysteine residue in the hinge region is substituted with a serine, and human IgG1 CH2 and CH3 domains, wherein the leucine at position 11 of the heavy chain variable region is substituted with a serine (9) G28-1 scFv VHL11S(SCC—P) H WCH2 WCH3 comprising a G28-1 scFv, an altered human IgG1 hinge in which the first cysteine is substituted with a serine, and human CH2 and CH3 domains, wherein the leucine at position 11 of the heavy chain variable region is substituted with a serine; (10) G28-1 scFv VH L115 mIgE CH2 CH3 CH4, comprising a G28-1 scFv and mouse IgE CH2, CH3 and CH4 regions, wherein the leucine at position 11 of the heavy chain variable region is substituted with a serine; (11) G28-1 scFv VH L115 mIgA WIgACH2 T4CH3, comprising a G28-1 scFv, a mouse IgA hinge, and a wild type IgA CH2 and a truncated IgA CH3 domain lacking the 4 carboxy amino acids GTCY (SEQ ID NO:265); (12) G28-1 scFv VHL11S hIgE CH2 CH3 CH4, comprising a G28-1 scFv and human IgE CH2, CH3 and CH4 regions, wherein the leucine at position 11 of the heavy chain variable region is substituted with a serine; and (13) G28-1 scFv VHL115 hIgAH WIgACH2 TCH3 comprising a G28-1 scFv, a portion of human IgA hinge, a wild type IgA CH2 and a truncated IgA CH3 domain lacking the 4 carboxy amino acids GTCY (SEQ ID NO:265), wherein the leucine at position 11 of the heavy chain variable region is substituted with a serine; all of which are herein incorporated by reference.

In preferred embodiments, CD37-specific SMIP polypeptides comprise humanized CD37-specific binding domains. In certain embodiments, the humanized CD37-specific SMIP polypeptides exhibit at least 70 percent identity (e.g., at least 70%, 72%, 74%, 76%, 80%, 82%, 84%, 85%, 86%, 88%, 90%, 92%, 94%, 95%, 96%, 97%, 98% or 99%) to the polypeptide set forth in SEQ ID NO:2 or 253, and specifically bind CD37. Exemplary humanized CD37-specific SMIP polypeptides comprise, consist essentially of, or consist of any amino acid sequence selected from the group consisting of SEQ ID NOS:6, 8, 10, 12, 14, 16, 18, 20, 22, 24, 26, 28, 30, 32, 34, 36, 38, 40, 42, 44, 46, 48, 52, 80, 82, 84, 86, 88, 222 and 262 but without the leader sequences, as well as SEQ ID NOS:247-254 and 266-269. Isolated nucleic acid molecules that encode exemplary humanized CD37-specific SMIP polypeptides provided herein include those that comprise SEQ ID NOS:5, 7, 9, 11, 13, 15, 17, 19, 21, 23, 25, 27, 29, 31, 33, 35, 37, 39, 41, 43, 45, 47, 51, 79, 81, 83, 85, 87, and 221.

In a preferred embodiment, a CD37-specific binding molecule comprises or consists of the amino acid sequence as set forth in SEQ ID NO:253. In another preferred embodiment, a CD37-specific binding molecule consists essentially of the amino acid sequence as set forth in SEQ ID NO:253. In yet another preferred embodiment, a CD37-specific binding molecule consists of the amino acid sequence as set forth in SEQ ID NO:253.

In certain embodiments, CD37-specific binding molecules are CD37-specific PIMS polypeptides. For example, a CD37-specific PIMS polypeptide may comprise from its amino- to carboxy-terminal orientation, a constant sub-region derived from an antibody (e.g., a region that comprises a CH2 domain and a CH3 domain of IgG, IgA or IgD, or a region that comprises a CH3 domain and a CH4 domain of IgM or IgE), a linker peptide and a CD37-specific binding domain (including a humanized CD37-specific binding domain). In certain embodiments, a CD37-specific PIMS polypeptide may further comprise a second linker peptide, which may or may not be the same as the linker peptide between the constant sub-region and the CD37-specific binding domain. The CD37-specific binding domain, the linker polypeptide, the immunoglobulin CH2 polypeptide, the immunoglobulin CH3 polypeptide, the immunoglobulin CH4 polypeptide are as described herein.

In certain embodiments, CD37-specific binding molecules are CD37-specific SCORPION polypeptides. For example, a CD37-specific SCORPION protein may be a single chain multivalent binding protein with an effector function, comprising from amino-terminus to carboxy-terminus: (a) a first binding domain comprising variable domains from an immunoglobulin or immunoglobulin-like molecule, (b) a first hinge or linker peptide, (c) an immunoglobulin constant sub-region providing an effector function, (d) a second hinge or linker peptide, and (e) a second binding domain comprising variable domains from an immunoglobulin or immunoglobulin-like molecule, wherein the first binding domain, the second binding domain, or both the first and second binding domains specifically bind to human CD37. The CD37-specific binding domain, the hinge or linker polypeptide, and the immunoglobulin constant sub-region are as described herein.

In further embodiments, an immunoglobulin Fc region (e.g., CH2, CH3, and/or CH4 regions) of CD37-specific binding molecules of the present disclosure may have an altered glycosylation pattern relative to an immunoglobulin reference sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831), such as N297 of the CH2 domain (EU numbering). Alternatively, the host cells producing fusion proteins of this disclosure may be engineered to produce an altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the Potelligent technology of BioWa/Kyowa Hakko is contemplated to reduce the fucose content of glycosylated molecules according to this disclosure. In one known method, a CHO host cell for recombinant immunoglobulin production is provided that modifies the glycosylation pattern of the immunoglobulin Fc region, through production of GDP-fucose.

Alternatively, chemical techniques are used to alter the glycosylation pattern of fusion proteins of this disclosure. For example, a variety of glycosidase and/or mannosidase inhibitors provide one or more of desired effects of increasing ADCC activity, increasing Fc receptor binding, and altering glycosylation pattern. In certain embodiments, cells expressing a CD37-specific binding molecule of the instant disclosure are grown in a culture medium comprising a carbohydrate modifier at a concentration that increases the ADCC of immunoglycoprotein molecules produced by said host cell, wherein said carbohydrate modifier is at a concentration of less than 800 μM. In a preferred embodiment, the cells expressing these multispecific fusion proteins are grown in a culture medium comprising castanospermine or kifunensine, more preferably castanospermine at a concentration of 100-800 μM, such as 100 μM, 200 μM, 300 μM, 400 μM, 500 μM, 600 μM, 700 μM, or 800 μM. Methods for altering glycosylation with a carbohydrate modifier such as castanospermine are provided in US Patent Application Publication No. 2009/0041756 or PCT Publication No. WO 2008/052030.

The present disclosure provides the use of mTOR or PI3K inhibitors in combination with any of the CD37-specific binding molecules described herein or known in the art for reducing B-cells or treating diseases or disorders associated with aberrant B-cell activity.

mTOR Inhibitors

By way of background, hyperproliferative diseases (such as cancer) can be due to aberrant cell signaling. For example, mammalian target of rapamycin (“mTOR”) is a large, multidomain serine/threonine kinase, which has a catalytic domain that has homology with the PI3K family of protein kinases. mTOR (also known as FK506 binding protein 12-rapamycin associated protein 1 or FRAP) is an important signaling intermediate molecule downstream of the PI3K/AKT pathway that inhibits apoptosis and functions as a sensor of nutrient and energy levels and redox status (see, e.g., Tokunaga et al. (2004) Biochem. Biophys. Res. Commun. 313:443; Grunwald et al. (2002) Cancer Res. 62:6141; Stolovich et al. (2002) Mol. Cell Biol. 22:8101). mTOR appears to be involved in cell growth, cell proliferation, cell motility, cell survival, protein synthesis, and transcription (see, e.g., Hay and Sonenberg (2004) Genes Dev. 18:1926; Beevers et al. (2006) Int. J. Cancer 119:757). The dysregulation of the mTOR pathway is implicated as a contributing factor to various human disease processes, especially various types of cancer (Beevers et al., 2006) that includes transformed B-cells (see Wlodarski et al. (2005) Cancer Res. 65:7800; Leseux et al. (2006) Blood 108:4156). The mTOR pathway has also been implicated in glioblastoma multiforme, renal cell carcinoma, and multiple myeloma.

mTOR exists in two complexes, mTOR Complex 1 (mTORC1) and mTOR Complex 2 (mTORC2) in cells (Wullschleger et al. (2006) Cell 124:471). mTORC1 is composed of mTOR, regulatory associated protein of mTOR (Raptor), mammalian LST8/G-protein β sub-unit like protein (mLST8/GβL) and PRAS40. This complex is characterized by the classic features of mTOR by functioning as a nutrient/energy/redox sensor and controlling protein synthesis.

mTORC1 regulates the activity of at least two proteins: P70S6 kinase 1 and 4E-BP1, the eukaryotic initiation factor 4E (eIF4E) binding protein 1. mTORC1 phosephorylates p70S6 kinase 1 at serine 389 and at threonine 412. This phosphorylation can be detected in whole cell extracts of growth factor-treated cells with an antibody specific for the phosphoserine 389 residue. mTORC1 has also been shown to phosphorylate at least four residues of 4E-BP1.

mTORC2 is composed of mTOR, rapamycin-insensitive companion of mTOR (Rictor), GβL, and mammalian stress-activated protein kinase interacting protein (mSIN1). mTORC2 has been shown to function as an important regulator of the cytoskeleton through its stimulation of F-actin stress fibers, paxillin, RhoA, Rac, Cdc42, and protein kinase Cα (PKCα). It phosphorylates the serine/threonine protein kinase AKT/PKB at serine residue 473.

As used herein, the term “mTOR inhibitor” refers to a compound or a ligand that inhibits at least one activity of an mTOR, such as the serine/threonine protein kinase activity on at least one of its substrates (e.g., p70S6 kinase 1, 4E-BP1, AKT/PKB and eEF2). A person skilled in the art can readily determine whether a compound, such as rapamycin or an analogue or derivative thereof, is an mTOR inhibitor. A specific method of identifying such compounds or ligands is disclosed in, for example, U.S. Patent Application Publication No. 2003/0008923.

In certain embodiments, an mTOR inhibitor inhibits at least one activity of mTORC1. In further embodiments, an mTOR inhibitor inhibits at least one activity of mTORC2. In still further embodiments, an mTOR inhibitor inhibits at least one activity of mTORC1 and at least one activity of mTORC2. In certain embodiments, an mTOR inhibitor is a compound or ligand that inhibits cell replication by blocking progression of the cell cycle from G1 to S by inhibiting the phosphorylation of serine 389 or threonine 412 of p70s6 kinase.

A preferred mTOR inhibitor, rapamycin (USAN generic name is sirolimus), is described in U.S. Pat. No. 3,929,992. In certain embodiments, a composition comprising a CD37-specific binding molecule can be combined or used in combination with an mTOR inhibitor, such as rapamycin (sirolimus), temsirolimus, deforolimus, everolimus, tacrolimus, zotarolimus, curcumin, farnesylthiosalicylic acid, or the like.

As used herein, the term “rapamycin analogue or derivative thereof” includes compounds having the rapamycin core structure as defined in U.S. Patent Application Publication No. 2003/0008923 (the rapamycin core structure is herein incorporated by reference), which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives.

Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462).

Examples of 27-esters and ethers of rapamycin are disclosed in U.S. Pat. No. 5,256,790, which is hereby incorporated by reference in its entirety.

Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference.

Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein.

Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican®, RAD-001 and SDZ-RAD. Another preferred mTOR inhibitor is zotarolimus, an antiproliferative agent (Abbott Laboratories). Zotarolimus is believed to inhibit smooth muscle cell proliferation with a cytostatic effect resulting from the inhibition of mTOR. Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, PROTOPIC® and PROGRAF®. Other preferred mTOR inhibitors include AP-23675, AP-23573, and AP-23841 (Ariad Pharmaceuticals).

Preferred rapamycin derivatives include everolimus, CCI-779 (rapamycin 42-ester with 3-hydroxy-2-(hydroxymethyl)-2-methylpropionic acid; U.S. Pat. No. 5,362,718); 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxy)ethyl rapamycin (U.S. Pat. No. 5,665,772).

Exemplary mTOR inhibitor compounds of Formula A provided in US 2008/0214596 (Novartis) are incorporated by reference herein. Compounds of Formula A are also disclosed, for example, in PCT Publication Nos. WO 94/09010, WO 95/16691, WO 96/41807, WO 99/15530, and in U.S. Pat. No. 5,362,718, which compounds are incorporated herein by reference. These compounds may be prepared using the procedures described in these references.

Additional mTOR inhibitors include TORC1 and TORC2 inhibitors. For example, OSI-027 (OSI Pharmaceuticals) is a small molecule TORC1/TORC2 inhibitor. OSI-027 inhibits both the TORC1 and TORC2 signaling complexes, allowing for the potential for complete truncation of aberrant cell signaling through this pathway. In addition, torkinibs, ATP-competitive mTOR kinase domain inhibitors and inhibitors of both mTORC1 and mTORC2 may also be used in combination with CD37-specific binding molecules according to the present disclosure. Exemplary torkinibs include PP242 and PP30 (see, Feldman et al. (2009) PLoS Biology 7:371) and Torin1 (Thoreen et al. (2009) J Biol Chem 284:8023).

### PI3K Inhibitors

Phosphoinositide 3-kinases (PI 3-kinases or PI3Ks) are a family of related intracellular single transducer enzymes capable of phosphorylating the 3 position hydroxyl group of the inositol ring of phosphatidylinositol (PtdIns or PI). These enzymes are also known as phosphatidylinositol-3-kinases. Based on based on primary structure, regulation, and in vitro lipid substrate specificity, the phosphoinositol-3-kinase family can be divided into three different classes: Class I, Class II and Class III (see Leevers et al. (1999) Current Op. Cell Biol. 11:219).

Class I PI3Ks are responsible for the production of phosphatidylinositol 3-phosphate (PI(3)P), phosphatidylinositor (3,4)-bisphosphate (PI(3,4)P₂) and phosphatidylinositol (3,4,5)-trisphosphate (PI(3,4,5)P₃. The PI3K is activated by G-protein coupled receptors and tyrosine kinase receptors. Class I PI3Ks are heterodimeric molecules composed of a regulatory and a catalytic subunit; they are further divided into IA and IB subsets on sequence similarity. Class IA PI3K are composed of one of five regulatory p85α, p55α, p50α, p85β or p55γ subunit attached to a p110α, β or δ catalytic subunit. The first two p110 isoforms (α and β) are expressed in all cells, but p110δ is primarily expressed in leukocytes. It has been suggested that p110δ evolved in parallel with the adaptive immune system. The regulatory p101 and catalytic p110γ subunits comprise the type IB PI3K.

PI3Ks have been linked to a diverse group of cellular functions, including cell growth, proliferation, differentiation, motility, survival and intracellular trafficking. Many of these functions relate to the ability of class I PI3Ks to activate protein kinase B (PKB, aka AKT). The class IA PI3K p110α is mutated in many cancers and many of these mutations cause the kinase to be more active. The PtdIns(3,4,5)P₃ phosphatase PTEN, which antagonizes PI3K signaling, is absent in many tumors. Hence, PI3K activity contributes to cellular transformation and the development of cancer. Reports suggest that p110α may play a role in cell survival, whereas p110β may be more important in promoting cell proliferation (see Benistant et al. (2000) Oncogene 19:5083). The p110δ and p110γ isoforms regulate different aspects of immune responses (Rommel et al. (2007) Nat. Rev. Immunol. 7:191; Ruckle et al. (2007) Nat. Rev. Drug Discov. 5:903). Isoform p100γ was suggested to play a key role as a modulator of inflammation and allergy (Wymann et al. (2003) Biochem. Soc. Trans. 31:275, while isoform p100δ was suggested to be critical for full B- and T-cell antigen receptor signaling (Okkenhaug et al. (2002) Science 297:1031). PI3Ks are also a key component of the insulin signaling pathway.

Class II PI3Ks comprise three catalytic isoforms (C2α, C2β, and C2γ), but, unlike Classes I and III, no regulatory proteins. Class II PI3Ks catalyze the production of PI(3)P and PI(3,4)P₂ from PI. C2α and C2β are expressed throughout the body; however, expression of C2γ is limited to hepatocytes. Some evidence has been presented that Class II PI3Ks, similar to Class I PI3Ks, can be activated by external stimuli via receptor tyrosine kinase (RTKs), cytokine receptors and integrins, suggesting a role in cancer, wound healing, and insulin signaling.

Class III PI3Ks produce only PI(3)P from PI, but are more similar to Class I in structure since they exist as a heterodimers having catalytic (Vps34) and regulatory (p150) subunits. Class III PI3Ks seem to be primarily involved in the trafficking of proteins and vesicles, phagosome maturation, and autophagy (Falasca et al. (2007) Biochem. Soc. Trans. 35:211).

The various 3-phosphorylated phosphoinositides that are produced by PI3Ks (e.g., PtdIns3P, PtdIns(3,4)P2, PtdIns(3,5)P2 and PtdIns(3,4,5)P3) function in a mechanism by which an assorted group of signaling proteins containing PX domain, pleckstrin homology domain (PH domains), FYVE domains and other phosphoinositide-binding domains are recruited to various cellular membranes through direct lipid-protein interactions (Fruman et al. (1998) Annu. Rev. Biochem. 67:481; Hawkins et al. (2006) Biochem. Soc. Trans. 34:647). For example, AKT is activated as a result of PI3-kinase activity because AKT requires the formation of the PtdIns(3,4,5)P3 (or “PIP3”) molecule in order to be translocated to the cell membrane. At PIP3, AKT is then phosphorylated by another kinase called phosphoinositide dependent protein kinase 1 (PDPK1), and is thereby activated. The “PI3K/AKT” signaling pathway has been shown to be required for a very diverse array of cellular activities—most notably cellular proliferation and survival.

In addition to AKT and PDK1, another related serine threonine kinase, SGK, is bound at the PIP3 molecule created as a result of PI3-kinase activity. PI3K has also been implicated in long term potentiation (LTP). The PI3K pathway also recruits many other proteins downstream, including mTOR, GSK3β, and PSD-95.

As used herein, the term “PI3K inhibitor” refers to a compound that inhibits at least one activity of a PI3K of Class I, II or III on at least one of its substrates (e.g., phosphorylating phosphatidylinositol to produce phosphatidylinositol 3-phosphate (PI(3)P), phosphatidylinositor (3,4)-bisphosphate (PI(3,4)P₂), or phosphatidylinositol (3,4,5)-trisphosphate (PI(3,4,5)P₃). A person skilled in the art can readily determine whether a compound, such as wortmannin or LY294002, is a PI3K inhibitor. A specific method of identifying such compounds or ligand is disclosed in, for example, U.S. Pat. Nos. 5,858,753; 5,882,910; and 5,985,589, Jackson et al. (2005) Nat. Med. 11:507, Pomel et al. (2006) J. Med. Chem. 49:3857; Palanki et al. (2007) J. Med. Chem. 50:4279), which methods are incorporated by reference herein.

In certain embodiments, a PI3K inhibitor inhibits the activity of a Class I PI3K. For example, a PI3K inhibitor may inhibit p110α, p110β, p110γ, or p110δ. In preferred embodiments, a PI3K inhibitor blocks or reduces the activity of p110γ or p110δ as compared to untreated p110γ or p110δ. In certain embodiments, a PI3K inhibitor inhibits the activity of a Class II PI3K. For example, a PI3K inhibitor may inhibit PI3K-C2α, PI3K-C2β, or PI3K-C2γ. In certain embodiments, a PI3K inhibitor inhibits the activity of a Class III PI3K, Vps34.

In certain embodiments, a PI3K inhibitor is selective or specific for a particular PI3K isoform. An inhibitor is “selective” or “specific” for a particular PI3K isoform if it inhibits the particular PI3K isoform more effectively than other PI3K isoforms. For example, an inhibitor specific for a particular PI3K isoform may have an IC₅₀ for the particular PI3K isoform at most about 1/10 (e.g., at most about 1/20, 1/30, 1/40, 1/50, 1/60, 1/80, 1/100, 1/200, 1/300, 1/400, 1/500, 1/600, 1/800, or 1/1000) of the IC₅₀ for other PI3K isoforms. For example, a p110δ-specific inhibitor may have an IC₅₀ value for p110δ at most about 1/10 of the IC₅₀ for other PI3K isoforms (e.g., p110α, p110β, or p110γ).

In preferred embodiments, a PI3K inhibitor is specific for p110α, p110β, p110γ, or p110δ. In certain embodiments, a PI3K inhibitor inhibits two or more classes or subclasses of PI3Ks. In certain embodiments, a PI3K inhibitor is also an mTOR inhibitor.

A preferred PI3K inhibitor is LY294002 (2-morpholin-4-yl-8-phenylchromen-4-one) or wortmannin. Both LY294002 and wortmannin are broad inhibitors against PI3K and can also inhibit mTOR. PI3K inhibitors useful in the combination therapy with CD37-specific binding molecules include wortmannin derivatives, such as PX-866 (see, Ihie et al., Mol Cancer Ther 3:763-72, 2004).

Exemplary p110γ-specific inhibitors useful in the present disclosure include furan-2-ylmethylene thiazolidinediones (AS-252424) (see, Pomel et al., 2006, supra) and 3,3′-(2,4-diaminopteridine-6,7-diyl)diphenol (see, Palanki et al., supra). Exemplary p110δ-specific inhibitors useful in the present disclosure include IC486068 and IC87114 (ICOS Corp., now Eli Lilly and Company) and CAL-101 and CAL-263 (Calistoga Pharmaceuticals). Another PI3K inhibitor that may be used in combination with a CD37-specific binding molecule is CAL-120, a PI3K inhibitor with p110δ and p110β inhibition (Calistoga Pharmaceuticals). Another exemplary PI3K inhibitor that may be used in combination with a CD37-specific binding molecule is GDC-0941 bismesylate (2-(1H-indazol-4-yl)-6-(4-methanesulfonyl-piperazin-1-ylmethyl)-4-morpholin-4-yl-thieno[3,2-d]pyrimidine, bimesylate salt), a p110α and P110δ selective inhibitor.

Additional PI3K inhibitors include pyrazole derivatives disclosed in PCT Publication No. WO 2009/059030, amino triazole derivatives disclosed in WO 2009/068482, an imidazothiadiazole compound disclosed in WO 2009/040552, fused pyrimidin-4-one compounds specific for p110δ disclosed in WO 2009/064802, morpholino-pyrimidine compounds that inhibit p110α disclosed in WO 2009/066084, a 4-pyrimidin-4-yl-morpholine derivative disclosed in WO 2009/042607, a 4-morpholin-4-yl-thienopyrimidine compound disclosed in WO 2009/036082, a pyridosulphonamide derivative disclosed in WO 2009/055418, pyridopyrimidine derivatives that inhibit p110α and/or p110γ disclosed in WO 2009039140, heterocyclic derivatives that inhibit p110α disclosed in WO 2009046448, furanopyrimidines and zolopyrimidines specific for p110δ disclosed in WO 2008/152394 and WO 2008/152390, quinazoline compounds specific for p110δ disclosed in WO 2008/152387, pyrimidine-substituted purine derivatives disclosed in WO 2009/045175, thienopyrimidine and pyrazolopyrimidine compounds disclosed in WO 2009/052145, imidazolopyrimidine, pyrrolopyrimidine and pyrazolopyrimidine analogues disclosed in WO 2009/070524, substituted imidazopyridazine disclosed in WO 2008/138834, thienopyrimidiene derivatives selective for the p110δ disclosed in WO 2009/053715, purine derivatives selective for the p110δ disclosed in WO 2009/053716, 2-(morpholin-4-yl)-substituted purine derivatives disclosed in WO 2009/045174, PI3Kδ (p110δ) inhibitors disclosed in U.S. Pat. Nos. 6,518,277 and 6,800,620 and U.S. Application Publication No. 2005/0261317, and BGT226, XL765 and BEZ235 (Novartis).

### Combinations and Pharmaceutical Compositions

The present disclosure provides combinations and pharmaceutical compositions that comprise CD37-specific binding molecules with mTOR inhibitors, PI3K inhibitors, or any combination thereof.

In certain embodiments, the present disclosure provides a CD37-specific binding molecule and an mTOR inhibitor. The CD37-specific binding molecule may be any one provided herein or known in the art, including CD37-specific antibodies, scFvs, Fabs, SMIPs, PIMS's and SCORPION polypeptides. An mTOR inhibitor may be any one known in the art or provided herein. For example, in certain embodiments, a combination or composition of the present disclosure comprises a CD37-specific antibody or SMIP protein and an mTOR inhibitor selected from sirolimus, temsirolimus, deforolimus, everolimus, tacrolimus, zotarolimus, curcumin, or farnesylthiosalicylic acid. In other preferred embodiments, the composition comprises a CD37-specific antibody whose light and heavy chains comprise SEQ ID NOS:307 and 308, respectively, or SEQ ID NOS:309 and 310, respectively, or a CD37-specific SMIP polypeptide comprising SEQ ID NO:6, 8, 10, 12, 14, 16, 18, 20, 22, 24, 26, 28, 30, 32, 34, 36, 38, 40, 42, 44, 46, 48, 52, 60, 80, 82, 84, 86, 88 or 253.

In certain preferred embodiments, a combination or composition of the present disclosure comprises (1) a CD37-specific antibody whose light and heavy chains comprise SEQ ID NOS:307 and 308, respectively and sirolimus, (2) a CD37-specific antibody whose light and heavy chains comprise SEQ ID NOS:307 and 308, respectively, and temsirolimus, (3) a CD37-specific antibody whose light and heavy chains comprise SEQ ID NOS:307 and 308, respectively, and everolimus, (4) a CD37-specific antibody whose light and heavy chains comprise SEQ ID NOS:307 and 308, respectively, and deforolimus, (5) a CD37-specific antibody whose light and heavy chains comprise SEQ ID NOS:307 and 308, respectively, and PP242, or (6) a CD37-specific antibody whose light and heavy chains comprise SEQ ID NOS:307 and 308, respectively, and PP30. In certain other preferred embodiments, a combination or composition of the present disclosure comprises (1) a CD37-specific antibody whose light and heavy chains comprise SEQ ID NOS:309 and 310, respectively and sirolimus, (2) a CD37-specific antibody whose light and heavy chains comprise SEQ ID NOS:309 and 310, respectively, and temsirolimus, (3) a CD37-specific antibody whose light and heavy chains comprise SEQ ID NOS:309 and 310, respectively, and everolimus, (4) a CD37-specific antibody whose light and heavy chains comprise SEQ ID NOS:309 and 310, respectively, and deforolimus, (5) a CD37-specific antibody whose light and heavy chains comprise SEQ ID NOS:309 and 310, respectively, and PP242, or (6) a CD37-specific antibody whose light and heavy chains comprise SEQ ID NOS:309 and 310, respectively, and PP30. In other preferred embodiments, a combination or composition of the present disclosure comprises (1) a CD37-specific SMIP polypeptide comprising SEQ ID NO:253 and sirolimus, (2) a CD37-specific SMIP polypeptide comprising SEQ ID NO:253 and temsirolimus, (3) a CD37-specific SMIP polypeptide comprising SEQ ID NO:253 and everolimus, (4) a CD37-specific SMIP polypeptide comprising SEQ ID NO:253 and deforolimus, (5) a CD37-specific SMIP polypeptide comprising SEQ ID NO:253 and PP242, or (6) a CD37-specific SMIP polypeptide comprising SEQ ID NO:253 and PP30.

In further embodiments, the present disclosure provides a CD37-specific binding molecule and a PI3K inhibitor. The CD37-specific binding molecule may be any one provided herein, including CD37-specific antibodies, scFvs, Fabs, SMIPs, PIMS's and SCORPION polypeptides. The PI3K inhibitor may be any one known in the art or provided herein. For example, in certain embodiments, a combination or composition of the present disclosure comprises a CD37-specific antibody or SMIP protein and a PI3K inhibitor selected from LY294002, wortmannin, p110γ-specific inhibitors and p110δ-specific inhibitors. In some of these embodiments, the composition comprises a CD37-specific antibody whose light and heavy chains comprise SEQ ID NOS:307 and 308, respectively, or SEQ ID NOS:309 and 310, respectively, or a CD37-specific SMIP polypeptide comprising SEQ ID NOS:6, 8, 10, 12, 14, 16, 18, 20, 22, 24, 26, 28, 30, 32, 34, 36, 38, 40, 42, 44, 46, 48, 52, 60, 80, 82, 84, 86, 88 or 253.

In certain embodiments, the composition of the present disclosure comprises (1) a CD37-specific antibody whose light and heavy chains comprise SEQ ID NOS:307 and 308, respectively, and LY294002, (2) a CD37-specific antibody whose light and heavy chains comprise SEQ ID NOS:307 and 308, respectively, and a p110γ-specific inhibitor, or (3) a CD37-specific antibody whose light and heavy chains comprise SEQ ID NOS:307 and 308, respectively, and a p110δ-specific inhibitor. In certain other embodiments, the composition of the present disclosure comprises (1) a CD37-specific antibody whose light and heavy chains comprise SEQ ID NOS:309 and 310, respectively, and LY294002, (2) a CD37-specific antibody whose light and heavy chains comprise SEQ ID NOS:309 and 310, respectively, and a p110γ-specific inhibitor, or (3) a CD37-specific antibody whose light and heavy chains comprise SEQ ID NOS:309 and 310, respectively, and a p110δ-specific inhibitor. In certain further embodiments, the composition of the present disclosure comprises a CD37-specific SMIP polypeptide comprising SEQ ID NO:253 and LY294002, or SEQ ID NO:253 and a p110γ-specific inhibitor, or SEQ ID NO:253 and a p110δ-specific inhibitor.

In certain embodiments, a CD37-specific binding molecule and an mTOR or PI3K inhibitor are formulated together in a solution or a suspension. In such formulations, the molar ratio of the CD37-specific binding molecule to the mTOR or PI3K inhibitor may be in the range of 1:1000 to 1000:1, such as 1:1000 to 1:500, 1:500 to 1:100, 1:100 to 1:10, 1:10 to 1:1, 1:5 to 5:1, 1:1 to 10:1, 10:1 to 1:10, 10:1 to 100:1, 100:1 to 500:1, or 500:1 to 1000:1.

Pharmaceutical compositions preferably comprise one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like. In addition, compounds may form solvates with water or common organic solvents. Such solvates are contemplated as well.

Suitable pharmaceutically acceptable carriers that may be used in the pharmaceutical compositions of the present disclosure include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Carriers used are chosen from, but not limited to, the above or combinations thereof, as appropriate, depending on the dosage form of the present disclosure.

Formulation of the pharmaceutical composition will vary according to the route of administration selected (e.g., solution, emulsion, tablets). For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., water, buffered water, 0.4% saline, 0.3% glycine, or aqueous suspensions may contain an active compound (e.g., a CD37-specific binding molecule and an mTOR or PI3K inhibitor) in admixture with excipients suitable for the manufacture of aqueous suspensions.

Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The binding molecule, inhibitor or combination compositions can be lyophilized for storage and reconstituted in a suitable carrier prior to use. This technique has been shown to be effective with conventional immunoglobulins. Any suitable lyophilization and reconstitution techniques can be employed. It will be appreciated by those skilled in the art that lyophilization and reconstitution can lead to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

In certain embodiments, the pharmaceutical compositions of the present disclosure may be in a form suitable for oral administration, such as in the form of a pill, capsule, solution or suspension. Such formulations may be prepared according to any method known to the art for producing oral formulations and may contain one or more agents including sweetening agents, flavoring agents, coloring agents and preserving agents. If in a tablet form, the compositions may comprise tablet excipients, such as a filler or diluent (e.g., calcium or sodium carbonate, lactorse, calcium or sodium phosphate), a disintegrant (maize starch or alginic acid), a binder (e.g., starch, gelatin or acacia), a glidant, a lubricant (e.g., magnesium stearate, stearic acid or talc), an anti-adherent, a flavor, or a colorant.

The concentration of a CD37-specific binding molecule or an mTOR or PI3K inhibitor in these formulations can vary widely, for example from less than about 0.5%, usually at or at least about 1%, to as much as 15 or 20% by weight and will be selected primarily based on fluid volumes, viscosities, etc., in accordance with the particular mode of administration selected. A typical pharmaceutical composition for parenteral injection could be made up to contain 1 mL sterile buffered water, and 50 mg of antibody. A typical composition for intravenous infusion could be made up to contain 250 mL of sterile Ringer's solution, and 150 mg of antibody. Actual methods for preparing parenterally administrable compositions are known or apparent to those skilled in the art and are described in more detail in, for example, Remington's Pharmaceutical Science, 15th ed., Mack Publishing Company, Easton, Pa. (1980).

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial or antifungal agents, for example, parabens, chlorobutanol, phenol, sorbic acid, thimerosal, or the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Compositions useful for administration may be formulated with uptake or absorption enhancers to increase their efficacy. Such enhancers include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

In addition, the properties of hydrophilicity and hydrophobicity of the compositions contemplated for use in the disclosure are well balanced, thereby enhancing their utility for both in vitro and especially in vivo uses, while other compositions lacking such balance are of substantially less utility. Specifically, compositions contemplated for use in the disclosure have an appropriate degree of solubility in aqueous media which permits absorption and bioavailability in the body, while also having a degree of solubility in lipids which permits the compounds to traverse the cell membrane to a putative site of action. Thus, antibody compositions contemplated are maximally effective when they can be delivered to the site of target antigen activity.

An exemplary composition of a CD37-specific binding molecule suitable for intravenous infusion comprises a CD37-specific antibody (e.g., an antibody whose light and heavy chains comprising SEQ ID NOS:307 and 308, respectively, or SEQ ID NOS:309 and 310, respectively) at a concentration range of about 0.5 to about 25 mg/ml, such as about 0.5 to about 2.5, about 2.5 to about 10, and about 10 to about 25 mg/ml.

An exemplary composition of a CD37-specific binding molecule suitable for subcutaneous administration comprises a CD37-specific antibody (e.g., an antibody whose light and heavy chains comprising SEQ ID NOS:307 and 308, respectively, or SEQ ID NOS:309 and 310, respectively) at a concentration range of about 25 to about 250 mg/ml, such as about 25 to about 100, and about 100 to about 250 mg/ml.

An exemplary composition of a CD37-specific binding molecule suitable for intravenous infusion comprises a CD37-specific SMIP polypeptide (e.g., a SMIP polypeptide comprising SEQ ID NO:253) at a concentration range of at a concentration range of about 0.5 to about 25 mg/ml, such as about 0.5 to about 2.5, about 2.5 to about 10, and about 10 to about 25 mg/ml.

An exemplary composition of a CD37-specific binding molecule suitable for subcutaneous administration comprises a CD37-specific SMIP polypeptide (e.g., a SMIP polypeptide comprising SEQ ID NO:253) at a concentration range of about 25 to about 250 mg/ml, such as about 25 to about 100, and about 100 to about 250 mg/ml.

An exemplary composition of an mTOR inhibitor is a solution that comprises rapamycin at a concentration range of 0.1 to 5 mg/ml (e.g., 1 mg/ml) suitable for oral administration. Another exemplary composition of an mTOR inhibitor is a tablet that comprises 0.1 to 5 mg (e.g., 0.1 to 0.5 mg, 0.5 to 1 mg, 1 to 2 mg, 2 to 3 mg, or 3 to 5 mg; or 0.25, 0.5, 1 or 2 mg) rapamycin suitable for oral administration. Another exemplary composition of an mTOR inhibitor is an oral solution that comprises rapamycin at a concentration range of about 0.1 to about 10 mg/ml (e.g., 0.1 to 0.5 mg/ml, 0.5 to 1 mg/ml, 1 to 5 mg/ml, or 5 to 10 mg/ml; or about 0.5, 1, or 2 mg/ml). Another exemplary composition of an mTOR inhibitor is a solution that comprises temsirolimus at a concentration range of 1 to 50 mg/ml (e.g., 1 to 5 mg/ml, 5 to 10 mg/ml, 10 to 20 mg/ml, 20 to 30 mg/ml, or 30 to 50 mg/ml; or 5, 10, or 25 mg/ml). Such a composition may be further diluted prior to administration via intravenous infusion. Another exemplary composition of an mTOR inhibitor is a tablet that comprises 1 to 25 mg (e.g., 1 to 2.5 mg, 2.5 to 5 mg, 5 to 10 mg, or 10 to 25 mg, or 1, 2.5, 5, 10, 15, 20, or 25 mg) everolimus suitable for oral administration. Other exemplary compositions of PI3K inhibitors are oral formulations of CAL-101, CAL-120 and CAL-263. Such oral formulations may comprise about 50 mg to about 500 mg, such as about 50 mg to about 100 mg, about 100 mg to about 200 mg, about 200 mg to about 300 mg, about 300 mg to about 400 mg, and about 400 mg to about 500 mg.

### Methods of Treatment

The present disclosure provides a method for reducing the number of B-cells or treating a disease or disorder associated with aberrant B-cell activity (e.g., B-cell cancers and autoimmune or inflammatory diseases) in a subject that has or is suspected of having the disease or disorder. The method comprises treating a subject with a CD37-specific binding molecule and an mTOR inhibitor, a CD37-specific binding molecule and a PI3K inhibitor, or any combination thereof. A combination of a CD37-specific binding molecule (e.g., anti-CD37 antibody or SMIP protein) and an mTOR or PI3K inhibitor can act synergistically to reduce the number of B-cells or to treat a disease or disorder associated with aberrant B-cell activity.

Two or more compounds that act synergistically interact such that the combined effect of the compounds is greater than the sum of the individual effects of each compound when administered alone (see, e.g., Berenbaum, Pharmacol. Rev. 41:93, 1989). For example, an interaction between a CD37-specific SMIP and another agent or compound may be analyzed by a variety of mechanistic and empirical models (see, e.g., Ouzounov et al., Antivir. Res. 55:425, 2002). A commonly used approach for analyzing the interaction between a combination of agents employs the construction of isoboles (iso-effect curves, also referred to as isobolograms), in which the combination of agents (dₐ, d_{b}) is represented by a point on a graph, the axes of which are the dose-axes of the individual agents (see, e.g., Ouzounov et al., supra; see also Tallarida, J. Pharmacol. Exp. Therap. 298:865, 2001).

Another method for analyzing drug-drug interactions (antagonism, additivity, synergism) known in the art includes determination of combination indices (CI) according to the median effect principle to provide estimates of IC₅₀ values of compounds administered alone and in combination (see, e.g., Chou. In Synergism and Antagonism Chemotherapy. Eds. Chou and Rideout. Academic Press, San Diego Calif., pages 61-102, 1991; CalcuSyn™ software). A CI value of less than one represents synergistic activity, equal to one represents additive activity, and greater than one represents antagonism.

Still another exemplary method is the independent effect method (Pritchard and Shipman, Antiviral Res. 14:181, 1990; Pritchard and Shipman, Antiviral Therapy 1:9, 1996; MacSynergy™ II software, University of Michigan, Ann Arbor, Mich.). MacSynergy™ II software allows a three-dimensional (3-D) examination of compound interactions by comparing a calculated additive surface to observed data to generate differential plots that reveal regions (in the form of a volume) of statistically greater than expected (synergy) or less than expected (antagonism) compound interactions. For example, a composition comprising a CD37-specific binding molecule and an mTOR or PI3K inhibitor will be considered to have synergistic activity or have a synergistic effect when the volume of synergy produced as calculated by the volume of the synergy peaks is preferably about 15% greater than the additive effect (that is, the effect of each agent alone added together), about 50% greater, preferably about a 2-fold to 10-fold greater, or preferably about a 3-fold to 5-fold or greater, than the additive effect.

In further embodiments, a CD37-specific binding molecule and an mTOR or PI3K inhibitor can be administered to act synergistically in the treatment of B-cell malignancies or B-cell cancers. Exemplary B-cell malignancies or B-cell cancers include B-cell lymphomas, such as various forms of Hodgkin's disease, non-Hodgkins lymphoma (NHL) or central nervous system lymphomas, small lymphocytic lymphoma, leukemias such as prolymphocytic leukemia, acute lymphoblastic leukemia (ALL), acute myeloid leukemia (AML), chronic lymphocytic leukemia (CLL), hairy cell leukemia and chronic myoblastic leukemia and myelomas (such as multiple myeloma). Additional B-cell cancers include small lymphocytic lymphoma, B-cell prolymphocytic leukemia, lymphoplasmacytic lymphoma (including Waldenström's macroglobulinemia), marginal zone lymphomas (including splenic marginal zone lymphoma and nodal marginal zone B-cell lymphoma), plasma cell myeloma/plasmacytoma, solitary plasmacytoma of bone, extraosseous plasmacytoma, nodal marginal zone lymphoma, extra-nodal marginal zone B-cell lymphoma of mucosa-associated (MALT) lymphoid tissue), follicular lymphoma, mantle cell lymphoma (MCL), diffuse large B-cell lymphoma, transforming large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

In certain embodiments, the B cell malignancy that the compounds, compositions, or combinations of the present disclosure may be used to treat is Burkitt's lymphoma. Burkitt's lymphoma (or “Burkitt's B cell malignancy”, or “Burkitt's tumor”, or “Malignant lymphoma, Burkitt's type”) is a cancer of the lymphatic system (in particular, B lymphocytes). It can be divided into three main clinical variants: the endemic, the sporadic and the immunodeficiency-associated variants.

Non-Burkitt's B cell malignancies that may be treated with the compounds, compositions, or combinations of the present disclosure include B-cell chronic lymphocytic leukemia (CLL)/small lymphocytic lymphoma, B-cell prolymphocytic leukemia, an acute lymphoblastic leukemia (ALL), lymphoplasmacytic lymphoma (including, but not limited to, Waldenström's macroglobulinemia), marginal zone lymphomas (including, but not limited to, splenic marginal zone B-cell lymphoma, nodal marginal zone lymphoma, and extranodal marginal zone B-cell lymphoma of mucosa-associated lymphoid tissue (MALT) type), hairy cell leukemia, plasma cell myeloma/plasmacytoma, follicular lymphoma, mantle cell lymphoma (MCL), diffuse large cell B-cell lymphoma, transforming large B cell lymphoma, mediastinal large B-cell lymphoma, intravascular large B-cell lymphoma, primary effusion lymphoma, and non-Hodgkins lymphoma (NHL).

Compositions and combination treatments of the instant disclosure are also useful in the treatment of disorders characterized by autoantibody production (e.g., autoimmune diseases). Autoimmune diseases include arthritis, rheumatoid arthritis, juvenile rheumatoid arthritis, osteoarthritis, polychondritis, psoriatic arthritis, psoriasis, dermatitis, polymyositis/dermatomyositis, inclusion body myostitis, inflammatory myositis, toxic epidermal necrolysis, systemic scleroderma and sclerosis, CREST syndrome, inflammatory bowel disease, Crohn's disease, ulcerative colitis, respiratory distress syndrome, meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, lupus, juvenile onset diabetes, multiple sclerosis, allergic encephalomyelitis, neuromyelitis, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis, (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic thrombocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre' Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, chronic obstructive pulmonary disease (COPD), amyotrophic lateral sclerosis (ALS), coronary artery disease, familial Mediterranean fever, microscopic polyangiitis, Cogan's syndrome, Whiskott-Aldrich syndrome and thromboangiitis obliterans, autoimmune thyroid disease (such as Graves' disease and Hashimoto's thyroiditis), Sjogren's syndrome, and idiopathic inflammatory myopathy (IIM), including dermatomyositis (DM) and polymyositis (PM).

Compositions or combination treatments of the instant disclosure are preferably used to treat B-cell lymphomas or leukemias such as B-cell non-Hodgkins lymphoma (NHL) (including Burkitt's lymphoma, chronic lymphocytic leukemia (CLL), small lymphocytic lymphoma (SLL), diffuse large B-cell lymphoma, follicular lymphoma, immunoblastic large cell lymphoma, precursor B-lymphoblastic lymphoma, and mantle cell lymphoma), hairy cell leukemia, B-cell pro-lymphocytic leukemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, and primary effusion lymphoma.

Further, compositions or combination treatments of the instant disclosure are preferably used to treat a disease characterized by autoantibody production such as idiopathic inflammatory myopathy, rheumatoid arthritis, juvenile rheumatoid arthritis, myasthenia gravis, Grave's disease, type I diabetes mellitus, anti-glomerular basement membrane disease, rapidly progressive glomerulonephritis, Berger's disease (IgA nephropathy), systemic lupus erythematosus (SLE), Crohn's disease, ulcerative colitis, idiopathic thrombocytopenic purpura (ITP), anti-phospholipid antibody syndrome, neuromyelitis optica, multiple sclerosis, an autoimmune disease, dermatomyositis, polymyositis, or Waldenström's macroglobinemia. In other preferred embodiments, compositions or combination treatments of the instant disclosure are used to treat a disease characterized by inappropriate T-cell stimulation associated with a B-cell pathway.

In certain instances, genetic lesions can be linked to or the cause of certain cancers. For example, cytogenetic analyses have revealed that mantle cell lymphoma (MCL) is closely associated with the t(11;14)(q13;q32) translocation (Rimokh et al., Genes Chromo. Cancer 2:223 (1990); Leroux et al., Br. J. Haematol. 77:346 (1991); Vandenberghe et al., Br. J. Haematol 81:212 (1992)). This translocation juxtaposes immunoglobulin heavy chain gene (IGH) sequences with the BCL-1 locus, leading to up-regulation of the CCND1 gene and consequently to an overexpression of cyclin D1 (de Boer et al., Cancer Res. 53:4148 (1993); de Boer et al., Oncogene 10:1833 (1995)). Overexpression of cyclin D1 is thought to be present in 100% of patients with MCL, but t(11;14)(q13;q32) is found in only 70% to 75% (Leroux et al., 1991; Vandenberghe et al., 1992). In addition, the frequency of this translocation in other cancers is 10-20% in B-prolymphocytic leukemia, plasma cell leukemia, and splenic lymphoma with villous lymphocytes, and 2-5% in chronic lymphocytic leukamia and in multiple myeloma (Huret, Atlas Genet. Cytogenet. Oncol. Haematol. (May 1998)). In further embodiments, the compositions of the instant disclosure are used to treat mantle cell lymphoma or multiple myeloma associated with chromosomal translocation t(11;14)(q13;q32) or cyclin D1 overexpression.

A method of the present disclosure includes steps of administration of a CD37-specific binding molecule and administration of an mTOR or PI3K inhibitor. In certain embodiments, the combination of compounds may be administered concurrently, together in the same pharmaceutically acceptable carrier, or separately (but concurrently). In other embodiments, the CD37 immunotherapeutic and mTOR or PI3K inhibitor can be administered sequentially (e.g., one, two, three, four, five, six, or seven days apart; one, two, three, or four weeks apart; or the like), in any order and in any combination.

The binding molecule, inhibitor or combination compositions may be administered orally, topically, transdermally, parenterally, by inhalation spray, vaginally, rectally, or by intracranial injection, or any combination thereof. When administered separately, a CD37-specific inhibitor and an mTOR or PI3K inhibitor may be administered by the same route or by different routes. For example, in one embodiment, the CD37-specific binding molecule is administered parenterally and the mTOR or PI3K inhibitor is administered orally, which can be concurrently or sequentially. The term “parenteral,” as used herein, includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient. Injection or infusion, especially intravenous, is preferred for administering a CD37-specific binding molecule.

In one embodiment, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or inhibitor) can be included in the formulations of the disclosure implanted near the cancer.

Pharmaceutical compositions may also be delivered to the patient at multiple sites. The multiple administrations may be rendered simultaneously or may be administered over a period of time. In certain cases it is beneficial to provide a continuous flow of the pharmaceutical composition. Additional therapy may be administered on a period basis, for example, hourly, daily, weekly or monthly.

Binding molecule, inhibitor, or combinations and compositions of this disclosure may comprise one or more than one binding molecule, inhibitor, or any combination thereof. Also contemplated by the present disclosure is the administration of binding molecule, inhibitor, or combinations and compositions in conjunction with a further therapeutic agent, such as pretreatment with steroids or acetaminophen. Further therapeutic contemplated by the disclosure are listed in paragraphs below.

A further therapeutic agent may be a B-cell-associated molecule. Other B-cell-associated molecules contemplated by the disclosure include binding molecules which bind to B-cell surface molecules that are not CD37. B-cell-associated molecules, include CD19 (B-lymphocyte antigen CD19, also referred to as B-lymphocyte surface antigen B4, or Leu-12), CD20 (CD20-specific binding molecules include TRU-015, rituximab, ofatumumab, ocrelizumab), CD21, CD22 (B-cell receptor CD22, also referred to as Leu-14, B-lymphocyte cell adhesion molecule, or BL-CAM), CD23, CD40 (B-cell surface antigen CD40, also referred to as Tumor Necrosis Factor receptor superfamily member 5, CD40L receptor, or Bp50), CD80 (T lymphocyte activation antigen CD80, also referred to as Activation B7-1 antigen, B7, B7-1, or BB1), CD86 (T lymphocyte activation antigen CD86, also referred to as Activation B7-2 antigen, B70, FUN-1, or BU63), CD137 (also referred to as Tumor Necrosis Factor receptor superfamily member 9), CD152 (also referred to as cytotoxic T-lymphocyte protein 4 or CTLA-4), L6 (Tumor-associated antigen L6, also referred to as Transmembrane 4 superfamily member 1, Membrane component surface marker 1, or M3S1), CD30 (lymphocyte activation antigen CD30, also referred to as Tumor Necrosis Factor receptor superfamily member 8, CD30L receptor, or Ki-1), CD50 (also referred to as Intercellular adhesion molecule-3 (ICAM3), or ICAM-R), CD54 (also referred to as Intercellular adhesion molecule-1 (ICAM1), or Major group rhinovirus receptor), B7-H1 (ligand for an immunoinhibitory receptor expressed by activated T-cells, B-cells, and myeloid cells, also referred to as PD-L1; see Dong, et al., “B7-H1, a third member of the B7 family, co-stimulates T-cell proliferation and interleukin-10 secretion,” Nat. Med., 5:1365-1369 (1999), CD134 (also referred to as Tumor Necrosis Factor receptor superfamily member 4, OX40, OX40L receptor, ACT35 antigen, or TAX-transcriptionally activated glycoprotein 1 receptor), 41 BB (4-1 BB ligand receptor, T-cell antigen 4-1BB, or T-cell antigen ILA), CD153 (also referred to as Tumor Necrosis Factor ligand superfamily member 8, CD30 ligand, or CD30-L), CD154 (also referred to as Tumor Necrosis Factor ligand superfamily member 5, TNF-related activation protein, TRAP, or T-cell antigen Gp39), Toll receptors, or the like.

Cytokines and growth factors are further therapeutic agents contemplated by this disclosure and include one or more of TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and erythropoietin. Pharmaceutical compositions or combinations in accordance with the disclosure may also include other known angiopoietins, for example Ang-1, Ang-2, Ang-4, Ang-Y, and/or the human angiopoietin-like polypeptide, and/or vascular endothelial growth factor (VEGF). Growth factors for use in pharmaceutical compositions of the disclosure include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurotrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Examples of chemotherapeutic agents contemplated as further therapeutic agents include alkylating agents, such as nitrogen mustards (e.g., mechlorethamine, cyclophosphamide, ifosfamide, melphalan, and chlorambucil); nitrosoureas (e.g., carmustine (BCNU), lomustine (CCNU), and semustine (methyl-CCNU)); ethyleneimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC)); antimetabolites, such as folic acid analogues (e.g., methotrexate, trimetrexate, and pemetrexed (multi-targeted antifolate)); pyrimidine analogues (such as 5-fluorouracil (5-FU), fluorodeoxyuridine, gemcitabine, cytosine arabinoside (AraC, cytarabine), 5-azacytidine, and 2,2′-difluorodeoxycytidine); purine analogues (e.g., 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; natural products, such as epipodophylotoxins (e.g., etoposide and teniposide); vinca alkaloids (e.g., vinblastine, vincristine, and vinorelbine); anti-tumor antibiotics such as actinomycin D, doxorubicin, and bleomycin; radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; methylhydrazine derivatives such as N-methylhydrazine (MIH) and procarbazine; and bifunctional compounds such as bendamustine (purine analog and alkylating agent).

Further therapeutic agents contemplated by this disclosure for treatment of autoimmune diseases are referred to as immunosuppressive agents, which act to suppress or mask the immune system of the individual being treated. Immunosuppressive agents include, for example, non-steroidal anti-inflammatory drugs (NSAIDs), analgesics, glucocorticoids, disease-modifying antirheumatic drugs (DMARDs) for the treatment of arthritis, or biologic response modifiers. Compositions in the DMARD description are also useful in the treatment of many other autoimmune diseases in addition to RA.

Exemplary NSAIDs are chosen from the group consisting of ibuprofen, naproxen, naproxen sodium, Cox-2 inhibitors such as Vioxx and Celebrex, and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD19, CD20, etc.), cytokine inhibitors, such as the TNF antagonists (e.g., etanercept (Enbrel®), adalimumab (Humira®) and infliximab (Remicade®)), chemokine inhibitors, and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline. In preferred embodiments, the anti-CD37 with mTOR or PI3K inhibitor compositions or combinations of this disclosure are used with methotrexate.

It is contemplated the CD37-specific binding molecule with mTOR or PI3K inhibitor composition or combination and the further therapeutic agent may be prepared and administered in the same formulation. Alternatively, each of the agents is administered as a separate formulation but concurrently (e.g., simultaneously or within a few minutes of each other), sequentially (e.g., with a delay of at least a few hours to a day or a week or more between administration of each agent), or any combination thereof.

In another aspect, the further therapeutic agent is administered prior to administration of the binding molecule, inhibitor, or combination composition. Prior administration refers to administration of the further therapeutic agent within the range of 10 or more minutes, hours, or one week prior to treatment with the binding molecule, inhibitor, or combination composition. It is further contemplated that the further therapeutic agent is administered subsequent to administration of the binding molecule composition. Subsequent administration is meant to describe administration within the range of 10 or more minutes, hours, or weeks after binding molecule, inhibitor, or combination composition treatment or administration.

It is further contemplated that when the binding molecule is administered in combination with a further therapeutic agent, wherein the further therapeutic agent is a cytokine or growth factor, or a chemotherapeutic agent, the administration may also include use of a radiotherapeutic agent or radiation therapy. The radiation therapy administered in combination with an antibody composition is administered as determined by the treating physician, and at doses typically given to patients being treated for cancer.

The combinations and pharmaceutical compositions of the present disclosure (e.g., combinations or compositions comprising a CD37-specific binding molecule, an mTOR or PI3K inhibitor, or a further therapeutic agent) are to be dosed and administered in a fashion (e.g., amounts, schedules and routes) consistent with good medical practice. Factors for consideration in this context include the particular disorder or disease being treated, the particular CD37-specific binding molecule, the particular mTOR or PI3K inhibitor, the particular further therapeutic agent (if any), the particular mammal being treated, the clinical condition of the individual patient, the site of delivery, the method of administration, the scheduling of administration and other factors known to medical practitioners.

The pharmaceutical compositions of the present disclosure may be administered in a single dose or in multiple doses. Standard dose-response studies, first in animal models and then in clinical testing, may be used to determine optimal dosages for particular disease states and patient populations.

In general, the initial therapeutically effective amount of a CD37-specific binding molecule, an mTOR or PI3K inhibitor, or a further therapeutic agent, when administered, for example via intravenous injection or infusion or via subcutaneous injection may be in the range of about 0.1 to 1000 mg/kg of patient body weight, such as about 0.1 to 1 mg/kg, about 1 to 10 mg/kg, about 10-50 mg/kg, about 50-100 mg/kg, about 100-500 mg/kg, or about 500-1000 mg/kg of patient body weight. The effective amounts of a CD37-specific binding molecule and an mTOR or PI3K inhibitor when used in combination therapy according to the present disclosure are less than the corresponding amounts when used alone. The administration of the CD37-specific binding molecule, the mTOR or PI3K inhibitor, or the further therapeutic agent may be repeated weekly, monthly, every three months, every six months, every year, or every two years at the same dose or at a dose different from the initial dose (e.g., three times, twice, two thirds, half, third, quarter of the initial dose), depending on the pharmacokinetic (PK) and pharmacodynamic (PD) properties, including absorption, distribution, metabolism, and excretion of the particular CD37-specific binding molecule, the particular mTOR or PI3K inhibitor, or the particular further therapeutic agent.

A dose of a CD37-specific binding molecule, an mTOR or PI3K inhibitor, or a further therapeutic agent when orally administered may be in the range of 0.1 to 1000 mg of the CD37-specific binding molecule, the mTOR or PI3K inhibitor, or the further therapeutic agent, such as 0.1 to 1 mg, 1 to 10 mg, 10 to 50 mg, 50 to 100 mg, 100 to 500 mg, or 500-1000 mg. A dose may be administered twice per day, once a day, once per week, once per month, or more or less frequently, also depending on the pharmacokinetic (PK) and pharmacodynamic (PD) properties, including absorption, distribution, metabolism, and excretion of the particular CD37-specific binding molecule, the particular mTOR or PI3K inhibitor, or the particular further therapeutic agent.

The administration of the binding molecule, inhibitor, or combination composition decreases the B-cell population by at least 20% after a single dose of treatment. In one embodiment, the B-cell population is decreased by at least about 20, about 30, about 40, about 50, about 60, about 70, about 80, about 90, or about 100%. B-cell reduction is defined as a decrease in absolute B-cell count below the lower limit of the normal range. B-cell recovery is defined as a return of absolute B-cell count to, for example, 70%, 80%, 90% of a subject's baseline value or normal range. Further, the administration of binding molecule, inhibitor, or combination composition of this disclosure results in desired clinical effects in the disease or disorder being treated.

In some embodiments, patients suffering from a B-cell cancer receive treatment according to the disclosure and demonstrate an overall beneficial response to the treatment, based on clinical criteria well-known and commonly used in the art, and as described below, such as a decrease in tumor size, decrease in tumor number or an improvement in disease symptoms.

Exemplary clinical criteria are provided by the U.S. National Cancer Institute (NCI), which has divided some of the classes of cancers into the clinical categories of “indolent” and “aggressive” lymphomas. Indolent lymphomas include follicular cell lymphomas, separated into cytology “grades,” diffuse small lymphocytic lymphoma/chronic lymphocytic leukemia (CLL), lymphoplasmacytoid/Waldenstrom's macroglobulinemia, Marginal zone lymphoma and Hairy cell leukemia. Aggressive lymphomas include diffuse mixed and large cell lymphoma, Burkitt's lymphoma/diffuse small non-cleaved cell lymphoma, Lymphoblastic lymphoma, Mantle cell lymphoma and AIDS-related lymphoma. In some cases, the International Prognostic Index (IPI) is used in cases of aggressive and follicular lymphoma. Factors to consider in the IPI include age (<60 years of age versus >60 years of age), serum lactate dehydrogenase (levels normal versus elevated), performance status (0 or 1 versus 2-4) (see definition below), disease stage (I or II versus III or IV), and extranodal site involvement (0 or 1 versus 2-4). Patients with 2 or more risk factors have less than a 50% chance of relapse-free and overall survival at 5 years.

Performance status in the aggressive IPI is defined as follows: Grade Description: 0 Fully active, able to carry on all pre-disease performance without restriction; 1 Restricted in physically strenuous activity but ambulatory and able to carry out work of a light or sedentary nature, e.g., light house work, office work; 2 Ambulatory and capable of all self-care but unable to carry out any work activities, up to and about more than 50% of waking hours; 3 Capable of only limited self-care, confined to bed or chair more than 50% of waking hours; 4 Completely disabled, unable to carry on any self-care, totally confined to bed or chair; and, 5 Dead (see The International Non-Hodgkin's Lymphoma Prognostic Factors Project. A predictive model for aggressive non-Hodgkin's lymphoma. N. Engl. J. Med. 329:987-94, 1993).

Generally, the grade of lymphoma is clinically assessed using the criterion that low-grade lymphoma usually presents as a nodal disease and is often indolent or slow-growing. Intermediate- and high-grade disease usually presents as a much more aggressive disease with large extranodal bulky tumors.

The Ann Arbor classification system can also be used to measure progression of tumors, especially non-Hodgkins lymphomas. For further details, see The International Non-Hodgkin's Lymphoma Prognostic Factors Project: A predictive model for aggressive non-Hodgkin's lymphoma, New England J. Med. (1993) 329:987. According to the Cheson criteria for assessing NHL developed in collaboration with the National Cancer Institute (Cheson et al., J Clin Oncol. 1999, 17:1244; Grillo-Lopez et al., Ann Oncol. 2000, 11:399), a complete response is obtained when there is a complete disappearance of all detectable clinical and radiographic evidence of disease and disease-related symptoms, all lymph nodes have returned to normal size, the spleen has regressed in size, and the bone marrow is cleared of lymphoma. Similar criteria have been developed for various other forms of cancers or hyperproliferative diseases and are readily available to a person of skill in the art. See, e.g., Cheson et al., Clin Adv Hematol Oncol. 2006, 4:4-5, which describes criteria for assessing CLL; Cheson et al., J Clin Oncol. 2003, 21:4642-9, which describes criteria for AML; Cheson et al., Blood 2000, 96:3671-4, which describes criteria for myelodysplastic syndromes.

In one aspect, a therapeutic effect of the methods according to the disclosure is determined by the level of response, for example a partial response is defined as tumor reduction to less than one-half of its original size. A complete response is defined as total elimination of disease confirmed by clinical or radiological evaluation. In one embodiment, the individual receiving treatment according to the disclosure demonstrates at least a partial response to treatment. In another aspect, an unconfirmed complete response is obtained when a patient shows complete disappearance of the disease and the spleen regresses in size, but lymph nodes have regressed by more than 75% and the bone marrow is indeterminate. An unconfirmed complete response meets and exceeds the criteria for partial response. An overall response is defined as a reduction of at least 50 percent in overall tumor burden.

In another aspect, a therapeutic response in patients having a B-cell cancer is manifest as a slowing of disease progression compared to patients not receiving therapy. Measurement of slowed disease progression or any of the above factors may be carried out using techniques well-known in the art, including bone scan, CT scan, gallium scan, lymphangiogram, MRI, PET scans, ultrasound, and the like.

In certain embodiments, the method of reducing the number of B-cells or treating a disease or disorder associated with aberrant B-cell activity in a subject having or suspected to have the disease or disorder comprises treating a subject with a combination of a CD37-specific binding molecule and an mTOR inhibitor. For example, the method may comprise administering to a subject in need thereof a CD37-specific binding molecule and an mTOR inhibitor selected from sirolimus, temsirolimus, deforolimus, everolimus, tacrolimus, zotarolimus, curcumin, or farnesylthiosalicylic acid. In some of the above embodiments, the CD37-specific binding molecule is a CD37-specific antibody or a CD37-specific binding molecule.

In further embodiments, the CD37-specific binding molecule is a CD37-specific antibody whose light and heavy chains comprise SEQ ID NOS:307 and 308, respectively, or SEQ ID NOS:309 and 310, respectively. In still further embodiments, the CD37-specific binding molecule is a CD37-specific SMIP polypeptide, such as a SMIP polypeptide comprising an amino acid sequence as set forth in SEQ ID NO:6, 8, 10, 12, 14, 16, 18, 20, 22, 24, 26, 28, 30, 32, 34, 36, 38, 40, 42, 44, 46, 48, 52, 60, 80, 84, 86, 88 or 253. In certain preferred embodiments, the combination is (1) a CD37-specific antibody whose light and heavy chains comprise SEQ ID NOS:307 and 308, respectively and sirolimus, (2) a CD37-specific antibody whose light and heavy chains comprise SEQ ID NOS:307 and 308, respectively, and temsirolimus, (3) a CD37-specific antibody whose light and heavy chains comprise SEQ ID NOS:307 and 308, respectively, and everolimus, (4) a CD37-specific antibody whose light and heavy chains comprise SEQ ID NOS:307 and 308, respectively, and deforolimus, (5) a CD37-specific antibody whose light and heavy chains comprise SEQ ID NOS:307 and 308, respectively, and PP242, or (6) a CD37-specific antibody whose light and heavy chains comprise SEQ ID NOS:307 and 308, respectively, and PP30. In certain other preferred embodiments, the combination is (1) a CD37-specific antibody whose light and heavy chains comprise SEQ ID NOS:309 and 310, respectively and sirolimus, (2) a CD37-specific antibody whose light and heavy chains comprise SEQ ID NOS: 309 and 310, respectively, and temsirolimus, (3) a CD37-specific antibody whose light and heavy chains comprise SEQ ID NOS: 309 and 310, respectively, and everolimus, (4) a CD37-specific antibody whose light and heavy chains comprise SEQ ID NOS: 309 and 310, respectively, and deforolimus, (5) a CD37-specific antibody whose light and heavy chains comprise SEQ ID NOS: 309 and 310, respectively, and PP242, or (6) a CD37-specific antibody whose light and heavy chains comprise SEQ ID NOS: 309 and 310, respectively, and PP30. In other preferred embodiments, the combination is (1) a CD37-specific SMIP polypeptide comprising SEQ ID NO:253 and sirolimus, (2) a CD37-specific SMIP polypeptide comprising SEQ ID NO:253 and temsirolimus, (3) a CD37-specific SMIP polypeptide comprising SEQ ID NO:253 and everolimus, (4) a CD37-specific SMIP polypeptide comprising SEQ ID NO:253 and deforolimus, (5) a CD37-specific SMIP polypeptide comprising SEQ ID NO:253 and PP242, or (6) a CD37-specific SMIP polypeptide comprising SEQ ID NO:253 and PP30.

In certain other embodiments, the method of reducing the number of B-cells or treating a disease or disorder associated with aberrant B-cell activity in a subject having or suspected to have the disease or disorder comprises treating a subject with a combination of a CD37-specific binding molecule and a PI3K inhibitor. For example, the method may comprise administering to a subject in need thereof a CD37-specific binding molecule and a PI3K inhibitor selected from LY294002, wortmannin, p110γ-specific inhibitors, or p110δ-specific inhibitors. In some of the above embodiments, the CD37-specific binding molecule is a CD37-specific antibody or a CD37-specific SMIP polypeptide.

In further embodiments, the CD37-specific binding molecule is a CD37-specific antibody whose light and heavy chains comprise SEQ ID NOS:307 and 308, respectively, or SEQ ID NOS:309 and 310, respectively. In still further embodiments, the CD37-specific binding molecule is a CD37-specific SMIP polypeptide, such as a SMIP polypeptide comprising an amino acid sequence as set forth in SEQ ID NO:6, 8, 10, 12, 14, 16, 18, 20, 22, 24, 26, 28, 30, 32, 34, 36, 38, 40, 42, 44, 46, 48, 52, 60, 80, 84, 86, 88 or 253. In certain preferred embodiments, the combination is (1) a CD37-specific antibody whose light and heavy chains comprise SEQ ID NOS:307 and 308, respectively, and LY294002, (2) a CD37-specific antibody whose light and heavy chains comprise SEQ ID NOS:307 and 308, respectively, and a p110γ-specific inhibitor, or (3) a CD37-specific antibody whose light and heavy chains comprise SEQ ID NOS:307 and 308, respectively, and a p110δ-specific inhibitor. In certain other preferred embodiments, the combination of the present disclosure is (1) a CD37-specific antibody whose light and heavy chains comprise SEQ ID NOS:309 and 310, respectively, and LY294002, (2) a CD37-specific antibody whose light and heavy chains comprise SEQ ID NOS:309 and 310, respectively, and a p110γ-specific inhibitor, or (3) a CD37-specific antibody whose light and heavy chains comprise SEQ ID NOS:309 and 310, respectively, and a p110δ-specific inhibitor. In other preferred embodiments, the combination is a CD37-specific SMIP polypeptide comprising an amino acid sequence as set forth in SEQ ID NO:253 with PI3K inhibitor LY294002, or SEQ ID NO:253 with a p110γ-specific inhibitor, or SEQ ID NO:253 with a p110δ-specific inhibitor.

In certain preferred embodiments, a CD37-specific binding molecule (e.g., a CD37-specific antibody whose light and heavy chains comprise SEQ ID NOS:307 and 308, respectively, or SEQ ID NOS: 309 and 310, respectively, or a CD37-specific SMIP comprising SEQ ID NO:253) is administered at a dosage ranging from about 0.03 mg/kg or about 20 mg/kg (e.g., 0.03 to 0.1 mg/kg, 0.1 to 0.5 mg/kg, 0.5 to 2.5 mg/kg, 2.5 to 5 mg/kg, 5 to 7.5 mg/kg, 7.5 to 10 mg/kg, 10 to 12.5 mg/kg, 12.5 to 15 mg/kg, 15 to 17.5 mg/kg, or 17.5 to 20 mg/kg) of the body weight of a subject per administration with an interval between administrations ranging from 1 day to 180 days (e.g., from 1 to 7 days, 1 to 14 days, 1 to 30 days, 1 to 60 days, 1 to 90 days, 1 to 120 days, 1 to 150 days; or daily, weekly, monthly, every 2 months, every 3 months, every 4 months, every 5 months, or every 6 months). In certain preferred embodiments, the CD37-specific binding molecule is intravenously administered (e.g., via intravenous infusion or injection). In certain other preferred embodiments, the CD37-specific binding molecule is subcutaneously administered.

In certain preferred embodiments, rapamycin is used as an mTOR inhibitor in combination with a CD37-specific binding molecule. Rapamycin may be orally administered with an initial dose of 1 to 15 mg (e.g., 1 to 2.5 mg, 2.5 to 5 mg, 5 to 7.5 mg, 7.5 to 10 mg, 10 to 12.5 mg, or 12.5 to 15 mg, or about 1, 2, 3, 4, 5, 6, 7, 8, 9, 10, 11, 12, 13, 14, or 15 mg) on day 1 followed by daily maintenance doses of 0.2 to 5 mg (e.g., 0.2 to 0.5 mg, 0.5 to 1 mg, 1 to 2 mg, 2 to 3 mg, 3 to 4 mg, or 4 to 5 mg; or 0.2, 0.4, 0.5, 0.6, 0.8, 1, 1.5, 2, 2.5, 3, 3.5, 4, 4.5 or 5 mg). In certain embodiments, rapamycin is orally administered with an initial dose of 6 mg on day 1 followed by daily maintenance doses of 2 mg. In certain other embodiments, rapamycin is orally administered with an initial dose of up to 15 mg on day 1 followed by daily maintenance doses of 5 mg.

In certain preferred embodiments, temsirolimus is used as an mTOR inhibitor in combination with a CD37-specific binding molecule. Temsirolimus may be administered via intravenous infusion at a dose of about 1 to 25 mg (e.g., 1 to 2.5 mg, 2.5 to 5 mg, 5 to 7.5 mg, 7.5 to 10 mg, 10 to 12.5 mg, 12.5 to 15 mg, 15 to 20 mg, or 20 to 25 mg, or about 1, 2, 3, 4, 5, 6, 7, 8, 9, 10, 11, 12, 13, 14, 15, 16, 17, 18, 19, 20, 21, 22, 23, 24, or 25 mg) once a week. In certain embodiments, temsirolimus is administered via intravenous infusion at a dose of 25 mg over a 30-60 minute period once a week.

In certain preferred embodiments, everolimus is used as an mTOR inhibitor in combination with a CD37-specific binding molecule. Everolimus may be orally administered daily at a dose of about 1 to about 10 mg (e.g., 1 to 2.5 mg, 2.5 to 5 mg, 5 to 7.5 mg, or 7.5 to 10 mg; or 1, 2, 2.5, 3, 4, 5, 6, 7, 7.5, 8, 9, or 10 mg). In certain embodiments, everolimus is orally administered daily at a dose of 5 mg. In certain embodiments, everolimus is orally administered daily at a dose of 10 mg.

In certain preferred embodiments, CAL-101, CAL-120 or CAL-263 is used as a PI3K inhibitor with a CD37-specific binding molecule. CAL-101, CAL-120 or CAL-263 may be orally administered twice or once daily at a dose range of about 10 to about 500 mg (e.g., 10 to 25 mg, 25 to 50 mg, 50 to 75 mg, 75 to 100 mg, 100 to 150 mg, 150 to 200 mg, 200 to 250 mg, 250 to 300 mg, 300 to 350 mg, 350 to 400 mg, 400 to 450 mg, or 450 to 500 mg; or 10, 20, 25, 30, 40, 50, 60, 70, 75, 80, 90, 100, 125, 150, 175, 200, 250, 300, 350, 400, 450, or 500 mg). In certain embodiments, CAL-101 is orally administered twice daily at a dose of 50 mg, 100 mg, 200 mg, or 350 mg per administration.

For a CD37-specific binding molecule and an mTOR or PI3K inhibitor to synergistically desirable effects (e.g., reducing or depleting B-cells or achieving clinical improvement), it is preferable that the CD37-specific binding molecule and the mTOR or PI3K inhibitor are simultaneously present in a subject that is treated. In certain preferred embodiments, a CD37-specific binding molecule is initially administered on the same day as an mTOR or PI3K inhibitor is administered. In certain other preferred embodiments, a CD37-specific binding molecule is initially administered within 30 days (e.g., 1, 2, 3, 4, 5, 6, 7, 10, 14, 21, or 30 days) prior to the initial administration of an mTOR or PI3K inhibitor. In certain further preferred embodiments, a CD37-specific binding molecule is initially administered within 30 days (e.g., 1, 2, 3, 4, 5, 6, 7, 10, 14, 21, or 30 days) subsequent to the initial administration of an mTOR or PI3K inhibitor.

### Kits

As an additional aspect, the disclosure includes kits which comprise one or more compounds or compositions useful in the methods of this disclosure packaged in a manner which facilitates their use to practice methods of the disclosure. In a simplest embodiment, such a kit includes a compound or composition described herein as useful for practice of a method of the disclosure packaged in a container such as a sealed bottle or vessel, with a label affixed to the container or included in the package that describes use of the compound or composition to practice the method of the disclosure. Preferably, the compound or composition is packaged in a unit dosage form. The kit may further include a device suitable for administering the composition according to a preferred route of administration or for practicing a screening assay. The kit may include a label that describes use of the binding molecule composition(s) in a method of the disclosure.

In certain embodiments, a kit of the present disclosure comprises a CD37-specific binding molecule and an mTOR or PI3K inhibitor packaged separate from one another as unit dosages or as independent unit dosages, with or without instructions that they be administered concurrently or sequentially. For example, a kit of the present disclosure suitable for treating a mantle cell lymphoma may comprise a unit dosage of a CD37-specific binding molecule (e.g., a CD37-specific antibody or a CD37-specific SMIP polypeptide) and a unit dosage of an mTOR inhibitor packaged separately. In certain embodiments, the kit may further comprise a further therapeutic agent (e.g., a CD20-specific binding molecule (such as TRU-015, rituximab, ofatumumab or ocrelizumab), cytokine, chemokine, growth factor, chemotherapeutic agent or radiotherapeutic agent) in another separate container.

In certain other embodiments, a CD37-specific binding molecule and an mTOR or PI3K inhibitor are formulated together. The resulting formulations may be packaged in unit-dose or multi-dose containers and may be stored in a freeze-dried (lyophilized) condition requiring only the addition of the sterile liquid carrier, such as water for injection immediately prior to use.

### EXAMPLES

### Example 1

### CD37-Specific Binding Molecules

Various CD37-specific binding proteins can be made with exemplary components provided herein. For example, CD37-specific antibodies or SMIP molecules can be made, and these molecules can be chimeric, humanized, or human. More specifically, preferred light chain variable region CDRs are found in SEQ ID NOS:236-240 and 247-254 and preferred heavy chain variable domain CDRs include SEQ ID NOS:241-245 and 247-254. Also, preferred light and heavy chain variable regions are provided in SEQ ID NOS:236-240 and SEQ ID NOS:241-245, respectively. Preferred light and heavy chain variable regions may also be found in SEQ ID NOS:247-254. Preferred variable domain linkers include SEQ ID NOS:225-229, while preferred hinges include SEQ ID NOS:230-235.

Preferred CD37-specific SMIP polypeptides include SEQ ID NOS:6, 8, 10, 12, 14, 16, 18, 20, 22, 24, 26, 28, 30, 32, 34, 36, 38, 40, 42, 44, 46, 48, 52, 80, 82, 84, 86, 88, 222 and 262 (but without the leader sequences) as well as SEQ ID NOS:247-254 and 266-269. A particularly preferred embodiment is CAS-024 [G28-1 VH (M99F, Y102S)-VL (T25A) scFv (SSC—P) H WCH2 WCH3], which is a recombinant, 483 amino acid single-chain fusion protein that binds to human CD37. The binding domain comprises a humanized scFv based on the G28-1 antibody variable region CDRs, including mutations in the heavy chain CDR3 and in the light chain CDR1. The variable domains are linked by a (G₄S)₅ (25 amino acid) sequence (SEQ ID NO:229), which is connected via a three amino acid junction (GDQ) to the amino terminus of a modified upper and core IgG1 hinge region (wherein the first two of three cysteines found in these hinge regions are each substituted with a serine). The carboxy-terminus of the hinge is fused to an effector domain comprising CH2 and CH3 domains of IgG1. The amino acid sequence of CAS-024 is set out in SEQ ID NO:253.

Preferred exemplary component parts of CD-37 specific SMIP molecules include leader sequences used for expression and export, but which are removed from the mature fusion protein when exported from a cell as set forth in SEQ ID NOS:223 and 224; linker sequences used to join light and heavy chain variable domains to form scFv binding domains as set forth in SEQ ID NOS:225-229; hinges used to join scFv binding domains to effector domains as set forth in SEQ ID NOS:230-235; light chain variable regions as set forth in SEQ ID NOS:236-240; heavy chain variable regions as set forth in SEQ ID NOS:241-245; and effector domains), as well as certain CD-37 specific SMIP molecules, including CAS-024 fusion protein, are provided in SEQ ID NOS:247-253.

### Example 2

### Growth Inhibition by CD37-Specific CAS-024 and Rapamycin Combination

CAS-024 [G28-1 VH (M99F, Y102S)-VL (T25A) scFv (SSC—P) H WCH2 WCH3] is described in Example 1. A nucleotide sequence encoding CAS-024 (including a leader sequence) is set forth in SEQ ID NO:221. Rapamycin (Sigma, St. Louis, Mo.) was dissolved in DMSO and stored at −20° C. until use. Human cell lines expressing CD37 used were Rec-1 (a Mantle Cell Lymphoma cell line) and SU-DHL-6 (Diffuse Large Cell Lymphoma cell line) (both from DSMZ, Braunschweig, Germany).

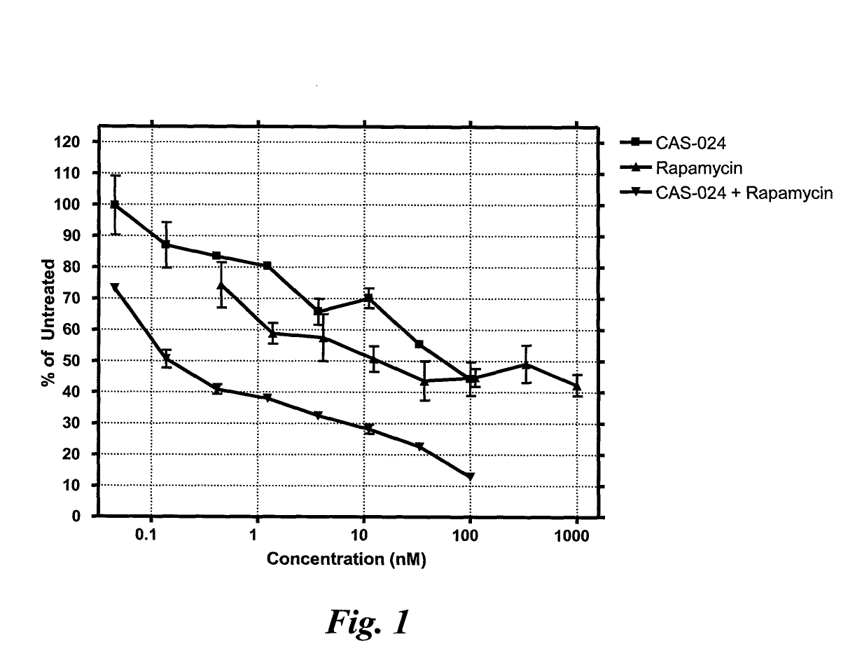

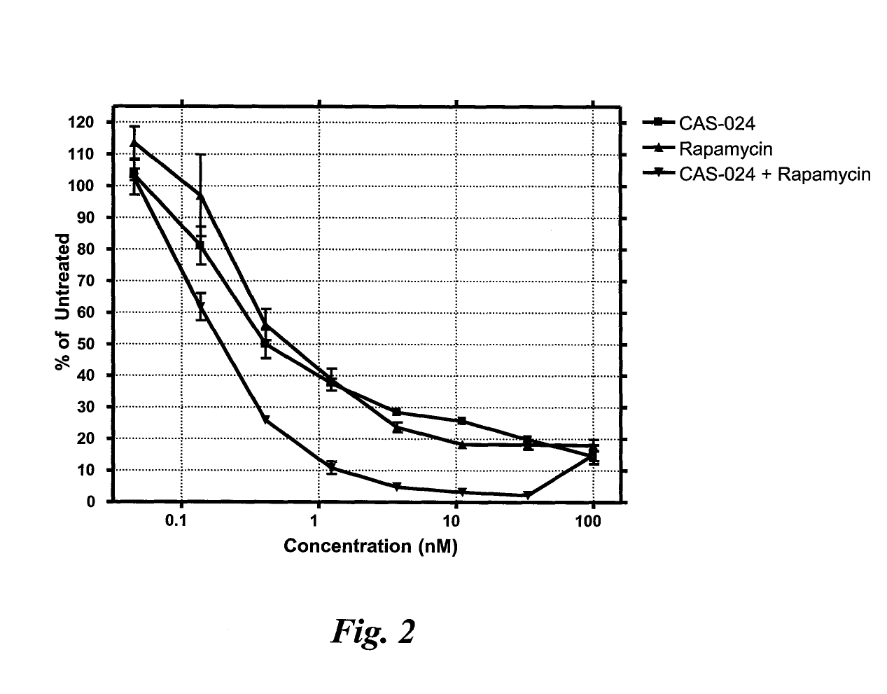

Rec-1 and SU-DHL-6 cells were plated at 1×10⁴ cells/well in 100 μL medium in 96-well plates. Cells were treated with various concentrations of CAS-024 (for concentrations, see FIGS. 1 and 2) that had been preincubated with anti-human IgG F(ab)′₂ and plates were incubated for 96 hr at 37° C., 5% CO₂ in the presence of serial dilutions of rapamycin. The final volume in each well was 150 μL. After incubation, plates were cooled to room temperature and labeled with 100 μL/well of ATPlite detection reagent (Perkin Elmer, Boston, Mass.). The assay measures cellular ATP as a marker for viable cells. Samples were analyzed by detection of luminescence using a Topcount NXT (Perkin Elmer, Waltham, Mass.) plate reader. Data were reduced using a 4-parameter curve fit in Prism (version 4.0, Graphpad Software, San Diego, Calif.) and the IC₅₀ defined as the concentration resulting in 50% inhibition compared to untreated cultures.

To determine whether these compounds were acting synergistically, the Median Effect/Combination Index (CI) method was used for data analysis (Chou and Talalay). A numerical value, assigned to each drug combination at predefined dose levels enables quantitative drug/drug interaction comparisons between different drug combinations. The CI values assign interactions into three categories: synergism, additivity, and antagonism (CI<1.0, =1, or >1.0, respectively). After labeling and data reduction, CI values were determined using the Calcusyn software package (Biosoft®, Cambridge, UK).

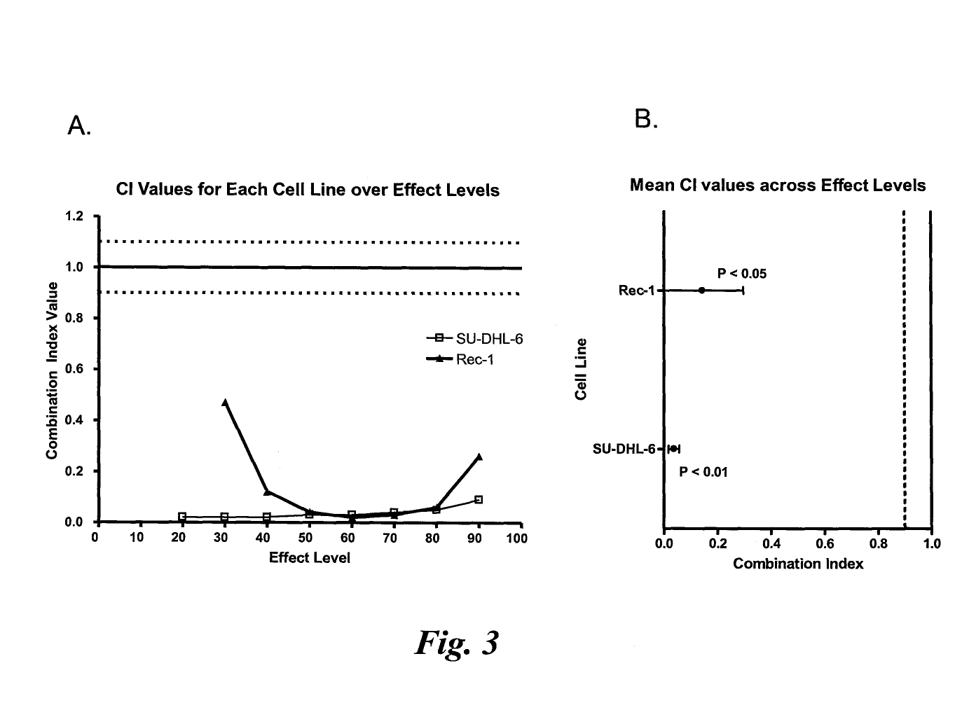

The combination of a CD37-binding molecule with mTOR inhibitor rapamycin inhibited Rec-1 (FIG. 1) and SU-DHL-6 (FIG. 2) cell growth more than either compound alone. Indeed, the measured CI of the CAS-024 and rapamycin were surprisingly strongly synergistic in cell growth inhibition (see FIG. 3).

### Example 3

### Growth Inhibition by CD37-Specific CAS-024 and Temsirolimus Combination

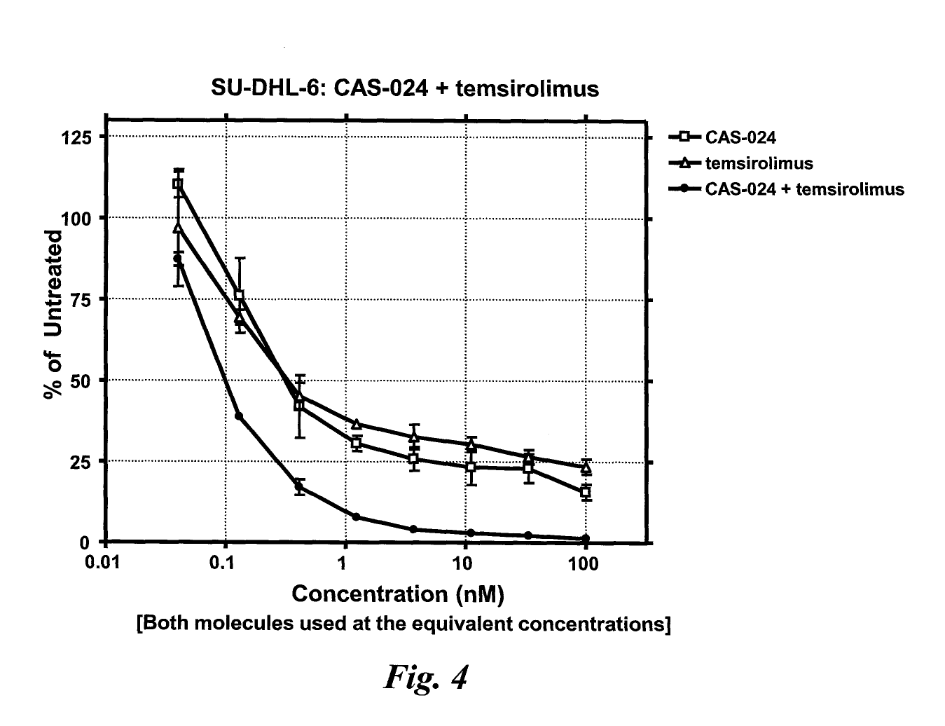

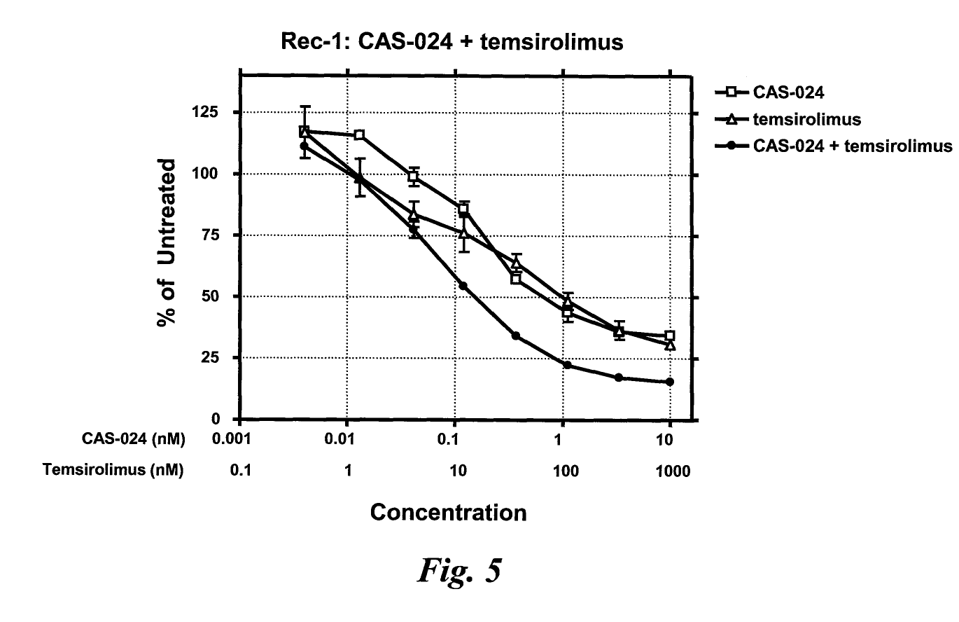

The effects of the combination of CAS-024 with another mTOR inhibitor, temsirolimus, on Rec-1 and SU-DHL-6 cell growth and the CI were determined using the methods as described in Example 2. The concentrations of CAS-024 and temsirolimus used are indicated in FIGS. 4 and 5.

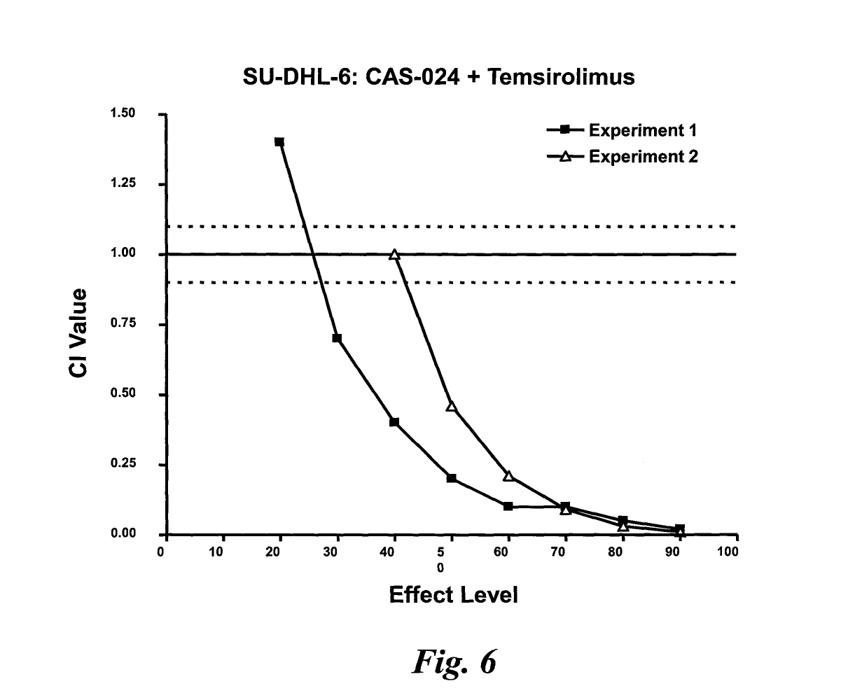

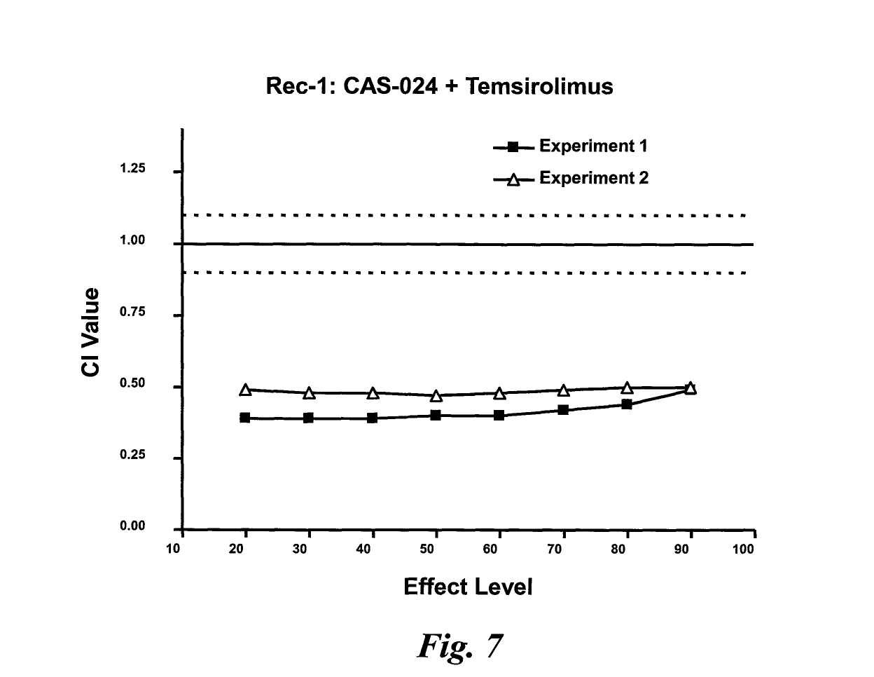

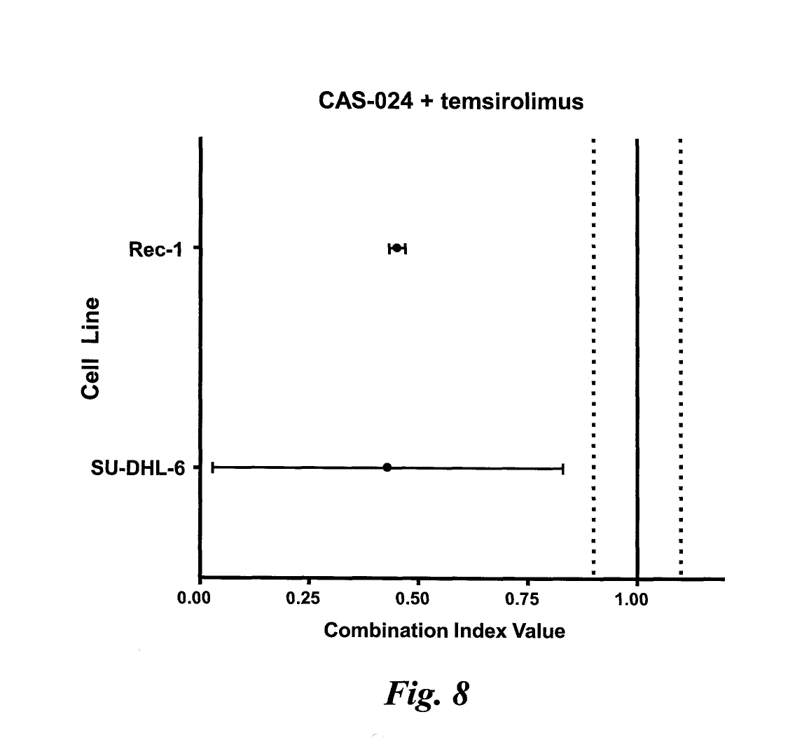

The results show that the combination of CAS-024 with temsirolimus inhibited SU-DHL-6 (FIG. 4) and Rec-1 (FIG. 5) cell growth more than either compound alone. The CI values measured show that CAS-024 in combination with temsirolimus synergistically inhibited SU-DHL-6 and Rec-1 cell growth (FIGS. 6-8).

### Example 4

### Growth Inhibition by CD37-Specific CAS-024 and LY294002 Combination

The effects of the combination of CAS-024 with a PI3K inhibitor, LY294002, on Rec-1 and SU-DHL-6 cell growth and the CI were determined using the methods as described in Example 2. The concentration ranges s of CAS-024 and LY294002 used are from 2 to 0.2 nM and from 50 to 0.4 μM, respectively.

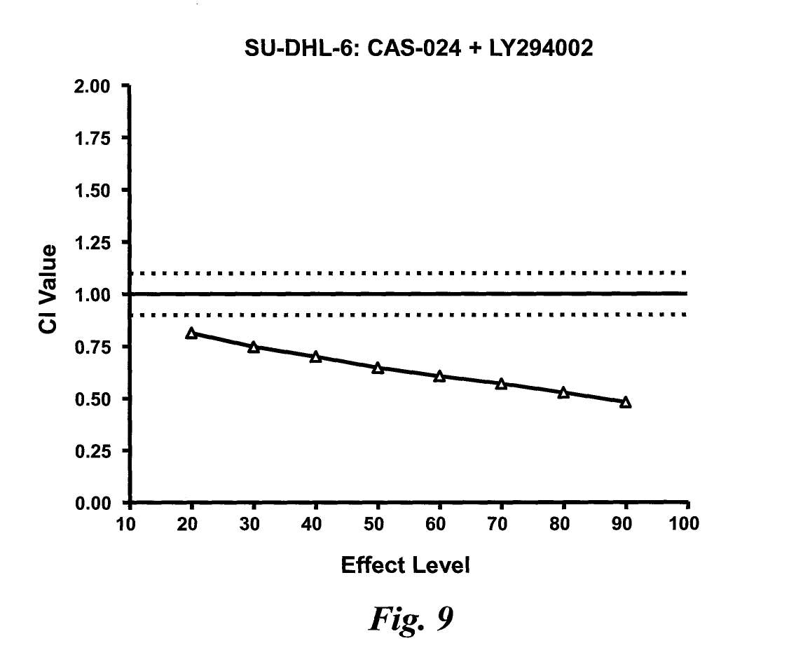

The results show that CAS-024 in combination with LY294002 synergistically inhibited SU-DHL-6 and Rec-1 cell growth (FIG. 9).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of reducing the number of B-cells or treating a disease or disorder associated with aberrant B-cell activity in a subject having or suspected having the disease or disorder, comprising treating a subject with a therapeutically effective amount of a CD37-specific binding molecule and a therapeutically effective amount of an mTOR or PI3K inhibitor.

2. The method of claim 1, wherein the method comprises administering to the subject an mTOR inhibitor.

3. The method of claim 2, wherein the mTOR inhibitor is sirolimus, temsirolimus, or a torkinib.

4. The method of claim 2, wherein the mTOR inhibitor is deforolimus, everolimus, tacrolimus, zotarolimus, curcumin, or farnesylthiosalicylic acid.

5. The method of claim 1, wherein the method comprises administering to the subject a PI3K inhibitor.

6. The method of claim 5, wherein the PI3K inhibitor is a P110δ-specific inhibitor.

7. The method of claim 1, wherein the CD37-specific binding molecule is a CD37-specific antibody or antigen-binding portion thereof, or a SMIP protein.

8. The method of claim 1, wherein the CD37-specific binding molecule is a humanized antibody or antigen-binding portion thereof, or a humanized SMIP protein.

9. The method of claim 1, wherein the CD37-specific binding molecule is a humanized CD37-specific SMIP protein comprising an amino acid sequence as set forth in SEQ ID NO:6, 8, 10, 12, 14, 16, 18, 20, 22, 24, 26, 28, 30, 32, 34, 36, 38, 40, 42, 44, 46, 48, 52, 60, 80, 82, 84, 86, or 88.

10. The method of claim 1, wherein the CD37-specific binding molecule is a humanized CD37-specific SMIP protein comprising an amino acid sequence set forth in SEQ ID NO:253.

11. The method of claim 1, wherein the CD37-specific binding molecule is a humanized CD37-specific antibody whose light and heavy chains comprise SEQ ID NOS:307 and 308, respectively, or SEQ ID NOS:309 and 310, respectively.

12. The method of claim 2, wherein the CD37-specific binding molecule comprises a SMIP protein having an amino acid sequence as set forth in SEQ ID NO:253, and wherein the mTOR inhibitor is sirolimus or temsirolimus.

13. The method of claim 2, wherein the CD37-specific binding molecule comprises an antibody whose light and heavy chains comprise SEQ ID NOS:307 and 308, respectively, or SEQ ID NOS:309 and 310, respectively, and wherein the mTOR inhibitor is sirolimus, temsirolimus, or a torkinib.

14. The method of claim 1, wherein the CD37-specific binding molecule and the mTOR or PI3K inhibitor are administered sequentially.

15. The method of claim 1, wherein the CD37-specific binding molecule and the mTOR or PI3K inhibitor are administered concurrently.

16. The method of claim 15, wherein the CD37-specific binding molecule and the mTOR or PI3K inhibitor are formulated together.

17. The method of claim 1, wherein the CD37-specific binding molecule is administered parenterally and the mTOR or PI3K inhibitor is administered orally.

18. The method according to claim 1, wherein the disorder or disease associated with aberrant B-cell activity is a B-cell lymphoma or leukemia, B-cell non-Hodgkins lymphoma (NHL), Burkitt's lymphoma, chronic lymphocytic leukemia (CLL), small lymphocytic lymphoma (SLL), diffuse large B-cell lymphoma, follicular lymphoma, immunoblastic large cell lymphoma, precursor B-lymphoblastic lymphoma, mantle cell lymphoma, hairy cell leukemia, Waldenström's macroglobulinemia, B-cell pro-lymphocytic leukemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, and primary effusion lymphoma; a disease characterized by autoantibody production, idiopathic inflammatory myopathy, rheumatoid arthritis, juvenile rheumatoid arthritis, myasthenia gravis, Grave's disease, type I diabetes mellitus, anti-glomerular basement membrane disease, rapidly progressive glomerulonephritis, Berger's disease (IgA nephropathy), systemic lupus erythematosus (SLE), Crohn's disease, ulcerative colitis, idiopathic thrombocytopenic purpura (ITP), anti-phospholipid antibody syndrome, neuromyelitis optica, multiple sclerosis, an autoimmune disease, dermatomyositis, polymyositis, or a disease characterized by inappropriate T-cell stimulation associated with a B-cell pathway.

19.-22. (canceled)

23. A composition, comprising:
(a) a CD37-specific binding molecule, and
(b) an mTOR or phosphatidylinositol 3-kinase (PI3K) inhibitor.

24. The composition of claim 23, wherein the composition comprises an mTOR inhibitor.

25. The composition of claim 24, wherein the mTOR inhibitor is sirolimus, temsirolimus, or a torkinib.

26. The composition of claim 23, wherein the mTOR inhibitor is deforolimus, everolimus, tacrolimus, zotarolimus, curcumin, or farnesylthiosalicylic acid.

27. The composition of claim 23, wherein the composition comprises a PI3K inhibitor.

28. The composition of claim 27, wherein the PI3K inhibitor is a p110δ-specific inhibitor.

29. The composition of claim 23, wherein the CD37-specific binding molecule is a CD37-specific antibody or antigen-binding portion thereof, or a SMIP protein.

30. The composition of claim 23, wherein the CD37-specific binding molecule is a humanized antibody or antigen-binding portion thereof, or a humanized SMIP protein.

31. The composition of claim 23, wherein the CD37-specific binding molecule is a humanized CD37-specific SMIP protein comprising an amino acid sequence as set forth in SEQ ID NO:6, 8, 10, 12, 14, 16, 18, 20, 22, 24, 26, 28, 30, 32, 34, 36, 38, 40, 42, 44, 46, 48, 52, 60, 80, 82, 84, 86, or 88.

32. The composition of claim 23, wherein the CD37-specific binding molecule is a humanized CD37-specific SMIP protein comprising an amino acid sequence set forth in SEQ ID NO:253.

33. The composition of claim 23, wherein the CD37-specific binding molecule is a humanized CD37-specific antibody whose light and heavy chains comprise SEQ ID NOS:307 and 308, respectively, or SEQ ID NOS:309 and 310, respectively.

34. The composition of claim 24, wherein the CD37-specific binding molecule comprises a SMIP protein with an amino acid sequence as set forth in SEQ ID NO:253, and wherein the mTOR inhibitor is sirolimus, temsirolimus, or a torkinib.

35. The composition of claim 24, wherein the CD37-specific binding molecule whose light and heavy chains comprise SEQ ID NOS:307 and 308, respectively, or SEQ ID NOS:309 and 310, respectively, and wherein the mTOR inhibitor is sirolimus, temsirolimus, or a torkinib.

36. The composition of claim 23, further comprising a CD20-specific binding molecule, a cytokine, a chemokine, a growth factor, a chemotherapeutic agent, or a radiotherapeutic agent.

37. The composition of claim 36, wherein the CD20-specific binding molecule is TRU-015, rituximab, ofatumumab, or ocrelizumab.

38. The composition of claim 36, wherein the chemotherapeutic agent is bendamustine.

39. The method according to claim 1, wherein the disorder is B-cell non-Hodgkins lymphoma (NHL).

40. The method according to claim 1, wherein the disorder is chronic lymphocytic leukemia (CLL).
